(19) United States
(12) Patent Application Publication
(10) Pub. No.: US 2025/0228473 A1
(43) Pub. Date: Jul. 17, 2025

(54) WEARABLE BIOSENSOR

(71) Applicant: Cornerstone Research Group, Inc., Miamisburg, OH (US)

(72) Inventors: John A. Hondred, I, Miamisburg, OH (US); Brian E. Henslee, Miamisburg, OH (US); Frank M. Zalar, Miamisburg, OH (US); Rebecca T. Browning, Miamisburg, OH (US)

(21) Appl. No.: 18/411,707

(22) Filed: Jan. 12, 2024

(51) Int. Cl. A61B 5/145; A61B 5/00; A61B 5/1486; A61B 10/00

(52) U.S. Cl. CPC A61B 5/14517; A61B 5/14546; A61B 5/1486; A61B 5/6833; A61B 10/0064; A61B 2562/125

(57) ABSTRACT

A biosensor system comprises a first layer comprising an adhesive layer, a second layer comprising an electrode layer (such as a laser induced graphene (LIG) electrode layer), a third layer comprising a microfluidic layer, and an electronic component. The microfluidic layer comprises reservoirs arranged in an array to chronologically capture and store liquid samples, e.g., sweat samples.

### CROSS REFERENCE TO RELATED APPLICATIONS

This application claims the benefit of U.S. Provisional Patent Application Ser. No. 63/479,953, filed Jan. 13, 2023, entitled “WEARABLE BIOSENSOR”, the disclosure of which is hereby incorporated by reference.

### STATEMENT REGARDING FEDERALLY SPONSORED RESEARCH OR DEVELOPMENT

This invention was made with U.S. Government support under Contract No. W81XWH-18-C-0120 and Contract No. W81XWH-19-C-0110 awarded by the U.S. Army Medical Research Acquisition Activity. The U.S. Government has certain rights in the invention.

### BACKGROUND

### Field of the Disclosure

Various aspects of the present disclosure relate generally to a wearable biosensor, and more particularly, to modular, wearable biosensor implemented as a sweat patch.

### Description of Related Art

Wearable sensors combine one or more sensors with embedded electronics in a package that is intended to be worn by a user. The wearable sensor is typically applied to a patient's skin and can be worn temporarily, e.g., for a few hours, or the wearable sensor can be worn by the user over an extended period of time. While the patch is applied to the patient's skin, the sensor combines with the embedded electronics to monitor the patient's physiological conditions, e.g., the patient's heart rate, body temperature, etc.

### BRIEF SUMMARY

According to aspects of the present invention, a wearable biosensor system comprises at least four layers. A first layer comprises an adhesive layer. A second layer comprises an electrode layer (e.g., such as a laser induced graphene (LIG) electrode layer). A third layer comprises a microfluidic layer. A fourth layer comprises an electronics layer, which supports an electronics system. The electrode layer (second layer) facilitates the real-time monitoring of a biological state or condition. Additionally, the microfluidic layer comprises reservoirs arranged in an array to chronologically capture and store liquid samples collected from an individual wearing the biosensor system.

According to further aspects herein, a microfluidic layer comprises an inlet, and an array of reservoirs arranged in series. The array of reservoirs are arranged such that at least a first reservoir in the array of reservoirs couples to the inlet. Also, each reservoir of the array of reservoirs includes at least one passive control valve to control filling of the corresponding reservoir. Moreover, each reservoir fills in series such that a first one of the reservoirs fills before filling a second one of the reservoirs.

According to yet further aspects herein, a hydrogel valve comprises a sodium acrylate hydrogel that is patterned in a microfluidic channel and is configured to swell when in contact with a liquid, closing off the channel.

Moreover, according to aspects herein, a biosensor device is provided. The biosensor device comprises a first layer that couples the biosensor device to skin of a user, and a microfluidic layer coupled to the first layer. The microfluidic layer sequentially captures and stores liquid collected from the skin of the user into discrete reservoirs. In particular, the discrete reservoirs include a first reservoir that collects a first sample of the liquid before a second reservoir collects a second sample of the liquid. Also, the microfluidic layer includes at least one valve that prevents the second sample collected into the second reservoir from contaminating the first sample collected into the first reservoir. The biosensor device also comprises a first sensor having laser induced graphene electrodes. The first sensor facilitates monitoring of a biological state or condition associated with the liquid collected from the skin of the user. The biosensor device further includes electronics coupled to the laser induced graphene electrodes of the first sensor that collects a real-time measurement of the biological state or condition.

According to still further aspects herein, another biosensor device is provided. The biosensor device comprises a first layer that couples the biosensor device to skin of a user, and a microfluidic layer coupled to the first layer. The microfluidic layer sequentially captures and stores liquid collected from the skin of the user into discrete reservoirs. The discrete reservoirs include a first reservoir that forms a serpentine pattern and collects a first sample of the liquid. The discrete reservoirs also include a second reservoir that forms a serpentine pattern and collects a second sample of the liquid. A first sensor comprises a first pair of laser induced graphene electrodes that align along the serpentine pattern of the first reservoir. Similarly, a second sensor comprises a second pair of laser induced graphene electrodes that align along the serpentine pattern of the second reservoir. The biosensor device also comprises electronics coupled to the first sensor and the second sensor. The electronics collect real-time measurements by detecting a change in impedance across the first pair of laser induced graphene electrodes as sweat flows over the first sensor. The electronics determine from the change in impedance measurements a flow rate of sweat into the first reservoir. Likewise, the electronics collect real-time measurements by detecting a change in impedance across the second pair of laser induced graphene electrodes as sweat flows over the second sensor. The electronics determine from the change in impedance measurements a flow rate of sweat into the second reservoir.

Additionally, according to aspects herein, yet another biosensor device is provided. The biosensor device comprises a first layer that couples the biosensor device to skin of a user, and a Polydimethylsiloxane (PDMS) microfluidic layer coupled to the first layer that collects sweat from the skin of the user. The microfluidic layer comprises an inlet, and an array of reservoirs coupled to the inlet. Each reservoir of the array of reservoirs includes at least one passive flow control capillary burst valve (CBV) to control filling of the corresponding reservoir. Also, each reservoir of the array of reservoirs includes a swelling hydrogel valve that contacts the sweat collected into the associated reservoir, swells and collapses an inlet channel thereof. Each reservoir fills in series such that a first one of the reservoirs fills before filling a second one of the reservoirs.

The biosensor device can also comprise a first sensor that monitors a biological state or condition associated with the liquid collected from the skin of the user. The biosensor device further comprises electronics coupled to the first sensor that collects a real-time measurement of the biological state or condition.

### BRIEF DESCRIPTION OF THE SEVERAL VIEWS OF THE DRAWINGS

FIG. 1 is an exploded schematic view of select components of a wearable biosensor according to aspects herein;

FIG. 2 is an exploded schematic view of another design for a wearable biosensor illustrating select components according to aspects herein;

FIG. 3 is an exploded schematic view of yet another design for a wearable biosensor illustrating select components according to aspects herein;

FIG. 4 is an exploded schematic view of select components of a two-part wearable biosensor according to aspects herein;

FIG. 5 is an example microfluidic layer having reservoir(s) for use with a wearable biosensor according to aspects herein;

FIG. 6 is an example schematic diagram of capillary burst valves to control reservoir filling according to aspects herein;

FIG. 7 is a graph showing an example of an internal pressure of a microfluidic device depicting different stages of filling of a corresponding control reservoir according to aspects herein;

FIG. 8 is an example laser induced graphene design for sweat rate sensing and electrochemical sensing according to aspects herein;

FIG. 9A is an example schematic of a check valve having a topper and flap for preventing contamination between reservoirs according to aspects herein;

FIG. 9B is an example of the check valve of FIG. 9A, showing the check valve in a closed state according to aspects herein;

FIG. 10A is an example perspective view of moisture swelling hydrogel plugs according to aspects herein;

FIG. 10B is an example view of moisture swelling hydrogel plugs of FIG. 10A in a first state, according to aspects herein;

FIG. 10C is an example view of moisture swelling hydrogel plugs of FIG. 10A in a second state, according to aspects herein;

FIG. 11A illustrates an example photo-patterned hydrogel sodium acrylate for closing a channel, according to aspects herein;

FIG. 11B illustrates an example fast acting hydrogel at a first time, according to aspects herein;

FIG. 11C illustrates an example fast acting hydrogel of FIG. 11B at a second time, according to aspects herein;

FIG. 11D illustrates an example fast acting hydrogel of FIG. 11B at a third time, according to aspects herein;

FIG. 11E illustrates an example fast acting hydrogel of FIG. 11B at a fourth time, according to aspects herein;

FIG. 12A illustrates a slow swelling hydrogel design, according to aspects herein;

FIG. 12B illustrates another example slow hydrogel design according to aspects herein;

FIG. 13A illustrates a fast swelling hydrogel valve design, according to aspects herein;

FIG. 13B illustrates another view of a fast swelling hydrogel valve design, according to aspects herein;

FIG. 13C is an example showing a hydrogel valve in a hydrogel valve chamber;

FIG. 13D is an example showing a hydrogel valve in a hydrogel valve chamber is a state where the hydrogel valve is starting to push a PDMS wall against an inlet of an associated reservoir;

FIG. 13E is an example showing a hydrogel valve in a hydrogel valve chamber in a fully swollen state sealing off an associated reservoir;

FIG. 14 is a flowchart illustrating communication between a smartphone and biosensor device;

FIG. 15A is an example screen shot of data exchanged with a biosensor; and

FIG. 15B is an example screen shot of data graphed by a smartphone showing flow rate of reservoirs of a biosensor according to aspects herein.

### DETAILED DESCRIPTION

Researchers are working to identify new biomarkers to help monitor cognition and stress in the human body and/or to enhance human performance. Traditional biomarkers, such as heart rate, temperature, oxygen partial pressure, blood glucose, electrolyte concentration, and others have been correlated with cognition and stress states. However, such correlations are indirect. By comparison, molecular biomarkers can be tailored to be more specific to cognition states, and can take the form of bioactive molecules including steroids, proteins, carbohydrates, lipids, and nucleic acids, etc. As such, there is a need to devise molecular biomarkers with stronger and more specific and/or direct links to cognition, stress, etc.

In this regard, aspects herein provide a modular, wearable, biosensor patch that is particularly well suited for monitoring cognition, stress, and other conditions. The biosensor patch may be durable but not bulky. Moreover, the wearable biosensor patch can provide real-time sweat monitoring, chronological sweat collection, or both. Moreover, where sweat collection is implemented, the biosensor patch can hold a large quantity of samples (e.g., up to or exceeding hundreds of microliters of sweat per patch), enabling a practical, wearable device that enables the collected sweat to be later extracted for post-analysis.

By way of example, a biosensor patch, as described more fully herein, can include real-time feedback, such as to monitor fatigue/stress of the wearer, e.g., through a lactate biosensor. The above-capabilities can be advantageous for health monitoring of personnel during physical training exercises or during active deployment of personnel, particularly where physical exertion, stress, or other sweat-inducing activity is required. The biosensor patch can also be used in commercial applications such as sports performance and training, health care, and other applications where an individual is expected to sweat.

As noted above, a biosensor patch, as described more fully herein, can include a sweat collection system. In an example implementation, sample quality and consistency are established by a system that implements the ability to collect timestamped samples of sweat. In practical applications, the biosensor patch is capable of holding a few microliters to hundreds of microliters of sweat per patch, which can later be extracted for post-analysis. Samples can be collected continuously, discretely, at varying time intervals (e.g., when an individual wearing the biosensor patch sweats), or combinations thereof. Also, in some embodiments, the patch can isolate sweat samples, e.g., for subsequent analysis. This type of collection system can help correlate wearable sensor readings with discrete volumes of sweat that can be quantified through more rigorous analytical techniques. As a result, the platform provides a means toward developing a reliable human performance sensor device.

In view of the above, aspects herein provide detector platforms as well as supporting electronics and fluidics for a low-profile wearable sensor device.

### Example Modular Biosensor Patch

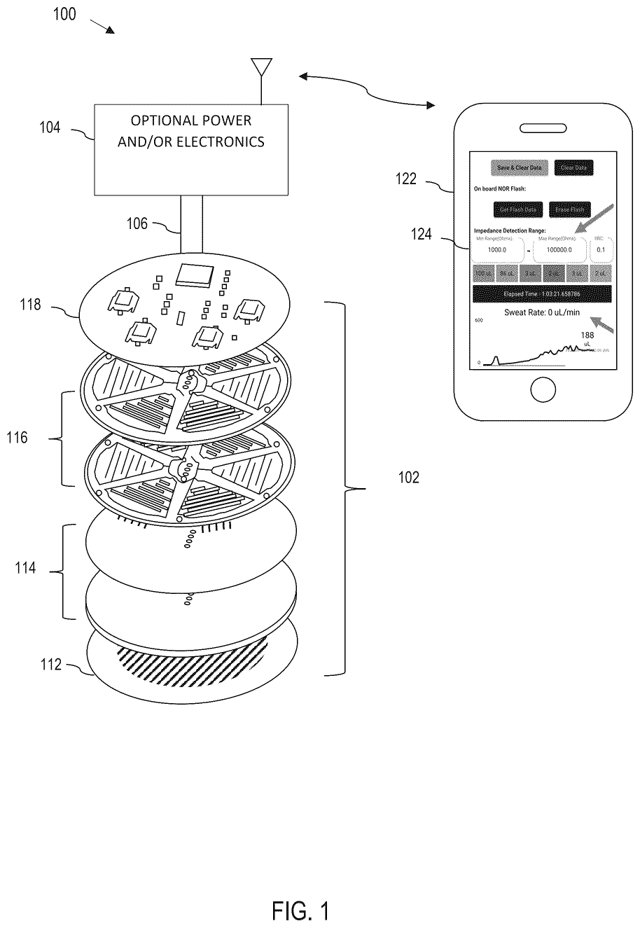

Referring now to the drawings, and in particular, to FIG. 1, an example wearable biosensor patch 100 is illustrated. The wearable biosensor patch 100 enables sweat aggregation, sweat analysis, sweat containment, or combinations thereof. In some embodiments, the wearable biosensor patch 100 provides real-time human sweat monitoring. According to further aspects herein, the wearable biosensor patch 100 is able to collect and chronologically store sweat, e.g., in discrete reservoirs. The chronological collection of sweat enables applications in biomarker sensing and human performance monitoring.

In some embodiments, the biosensor patch 100 is implemented as a stackable structure 102 that can connect to an optional secondary device 104 via a suitable interconnect, e.g., a ribbon cable 106. The secondary device 104 can enclose battery power, optional electronics or additional electronics (e.g., processor, memory, wireless transceiver, etc.), and/or a combination thereof, etc. In this regard, the stackable structure 102 can define a disposable component, whereas the secondary device 104 can define a reusable component.

In general, the wearable biosensor patch 100 is implemented as a structure 102 that can include a (medical grade) adhesive layer 112, one or more laser induced graphene (LIG) electrode layers 114, one or more Polydimethylsiloxane (PDMS) microfluidic layers 116, an optional flexible electronic layer 118, or any combination thereof. Moreover, features of each layer can be combined or comingled into a single layer. For instance, one layer can include both microfluidics (e.g., for sweat collection) and LIG electrodes for sweat measurements.

For instance, in the illustrated implementation, the wearable biosensor patch 100 is carried out using a multilayer stackable structure 102 that includes a (medical grade) adhesive layer 112, one or more laser induced graphene electrode layers 114, one or more Polydimethylsiloxane (PDMS) microfluidic layers 116, and an optional flexible electronic layer 118, each in separate layers. In example implementations, these layers are laminated together to create a disposable sweat patch that can connect to an electronic board (e.g., within the secondary device 104). In practice, as noted above, layers can be combined. For instance, the LIG electrodes can be combined with the microfluidics, the electronics, or both on a single layer. The layers are illustrated as separate for clarity of discussion. As such, the wearable biosensor patch 100 includes an adhesive layer, one or more LIG electrodes, at least one microfluidics layer, and electronics, combined in any practical manner.

Moreover, in some implementations, the biosensor patch 100 (e.g., via the optional secondary device 104 or via the optional flexible electronic layer 118, wirelessly communicates collected data, e.g., via Bluetooth, to a remote computer device 122, such as a smartphone, remote server, other computer, etc.

In an example implementation, a small and compact reusable electronic board (e.g., within the optional secondary device 104) connects to the disposable sweat patch multilayer stackable structure 102, and includes a small potentiostat chip for collecting measurements. This reusable board within the optional secondary device 104 further wirelessly communicates with a custom remote smartphone app 124 running on the remote computer device 122 (e.g., a smartphone), e.g., via Bluetooth. Here, the biosensor patch 100 efficiently collects sweat and chronologically stores sweat in individual reservoirs.

Additionally, the electronic board of the secondary device 104 communicates via Bluetooth to the corresponding smartphone app 124. The smartphone app 124 receives data from the biosensor patch 100, and accurately records and displays sweat data, e.g., volume of collected sweat, sweat rate, an amperometric response to lactate, combination thereof, etc. In an example implementation, the smartphone app 124 displays the amount of sweat collected by the biosensor patch 100, calculates the instantaneous sweat rate, or both, examples of which are described more fully herein. In some configurations, sweat from each of the reservoirs is collected with greater than 90% collection efficiency and presents no signs of cross-contamination.

In practical implementations, the biosensor patch 100 is manufactured in a compact 2 in (approximately 5.08 cm) diameter and <0.125 in (0.3175 cm) thick form factor. In some implementations, the wearable biosensor patch 100 can hold a few microliters to hundreds of microliters of sweat per patch, which can later be extracted for post-analysis. The patch is less bulky than similar devices, can hold more sweat, and can optionally provide real-time feedback to monitor fatigue/stress, e.g., through a lactate biosensor.

The adhesive layer 112 comprises an adhesive that does not interfere with the sample collection and analysis, yet is sufficiently adhesive to hold the wearable biosensor patch 100 on the user, e.g., adhere the biosensor patch 100 to the arm of the user, for the duration of sample collection and/or analysis, e.g., typically a few hours up to a few days.

The LIG electrode layer(s) 114 each implement at least one sensitive sweat rate sensor that is formed, for instance, by one or more electrodes positioned on a support layer. In an example configuration, as sweat flows over an LIG electrodes, the impedance of the LIG electrode decreases. Change of impedance of the LIG electrodes can be correlated to volumetric flow rate, amount of sweat collected by the patch, instantaneous sweat rate, etc. In this regard, real-time monitoring can be carried out without delays that might otherwise be required by optical or imaging solutions that require additional time for analysis. In some implementations, this sweat rate sensor will allow, for example, the user to time-stamp the collected sweat to evaluate how biomarker concentrations changed during the different user experiences/events.

In practice, the electrode layer(s) 114 can include a variety of electrochemical sensors utilizing LIG electrodes. For instance, a three-LIG electrode sensor could be constructed into a variety of electrochemical sensors (e.g. impedance, amperometric, potentiometric, or a combination thereof). Alternatively, and/or additionally, such sensor(s) can be functionalized with different biomolecules to create different biosensors. As an example, modular biosensor functionality can be realized by fabricating a sensitive lactate sensor that can be used to monitor the transition from aerobic to anaerobic activity.

As will be described in greater detail herein, in some embodiments, the microfluidic layer(s) 116 carry out sweat collection using large reservoirs arranged in arrays. This configuration can collect a few microliters to hundreds of microliters of sweat with reliable and complete filling of the reservoirs. In some implementations, soft lithography casting techniques are utilized to produce the microfluidic devices. In some implementations, Polydimethylsiloxane (PDMS) is utilized to fabricate microfluidics, due to its biocompatibility, flexibility, chemical stability, and transparency.

In some configurations, the PDMS microfluidics is flexible enough to conform to an individual for a wearable application and elastic enough to be easily peeled off the mold without tearing. Additionally, the PDMS is sufficiently cross-linked and/or cured such that the microfluidic channels and the reservoir geometry will not change significantly while bending, so as to avoid inconsistencies in fluid flow and different amounts of sweat that is stored, respectively. This eliminates large inconsistences in the sweat rate sensor as it quantifies the fluid flow based on the geometry of the reservoir.

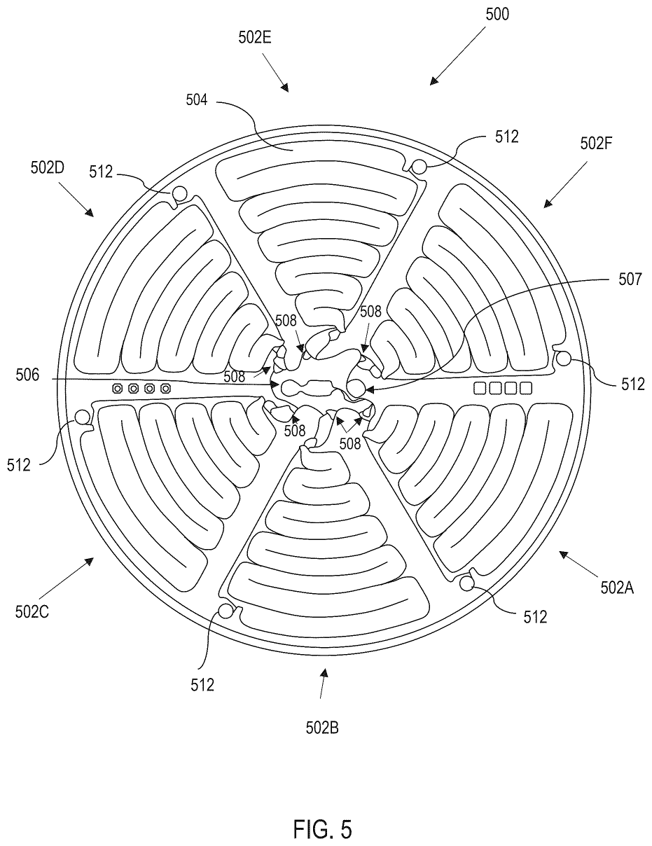

In some implementations, sample collection into reservoirs is carried out using passive flow control capillary burst valves (CBV). By way of introduction, capillary burst valves passively control the fluid flow of sweat in the microfluidic device. Moreover, the capillary burst valves feature a geometry that “bursts” after reaching an internal pressure, e.g., which can be defined by the height, width, shape of the valve, or a combination thereof. Utilizing the capillary burst valves, the sweat patch is able to store sweat in different reservoirs, e.g., six reservoirs in an example implementation. Moreover, the sweat can be efficiently extracted from the reservoirs for post analysis, examples of which are described in greater detail herein, with regard to FIG. 5.

In addition to or in lieu of capillary burst valves, a unique approach is provided for isolating the sweat collection reservoir using fast-swelling hydrogel valves. After filling the reservoir, sweat comes into contact with a hydrogel valve that swells, collapsing the inlet channel of the reservoir. This hydrogel valve effectively prevents cross-contamination between the different reservoirs.

The electronic layer 118 includes a low power electronic system design that, in some implementations, can achieve sub-microampere resolution from an unspecialized organic FET (OFET) device in contact with synthetic eccrine sweat. This same circuit is capable of supporting device modularity to different types of sensors, including electrochemical sensor probes for sweat analytes. Wide area sweat aggregation can transition into a flexible microfluidic device.

In some embodiments, the electronics layer 118 is re-usable whereas the remainder of the layers (e.g., the medical grade adhesive layer 112, one or more laser induced graphene electrode layers 114, and one or more Polydimethylsiloxane (PDMS) microfluidic layers 116), define a disposable patch. In other embodiments, the secondary device 104 (e.g., battery power and/or electronics) is reusable whereas the entire stackable structure 102 is disposable. In this configuration, the disposable stackable structure 102 can include an electronics layer, e.g., for connection to the ribbon cable 106, for signal conditioning or processing, for calibration, etc. Moreover, in some configurations, the ribbon cable 106 can define part of the disposable, stackable structure 102, whereas in other configurations, the ribbon cable forms part of the reusable structure.

In some embodiments, such as where only real-time monitoring of an active level is desired, the stackable structure can include the one or more laser induced graphene electrode layers 114, and omit or simplify the one or more Polydimethylsiloxane (PDMS) microfluidic layers 116), such that the sequential, serial collection of samples becomes optional or omitted altogether.

Conversely, where sample collection is desired, but real-time monitoring is not required, aspects herein can include the one or more Polydimethylsiloxane (PDMS) microfluidic layers 116) and omit or simplify the electrode layer(s) 114.

In some embodiments, electronics (e.g., within the electronic layer 118 and/or optional secondary device 104) communicate, e.g., via Bluetooth, to a remote computer device 122 (such as a smartphone). This enables, for example, the creation and/or use of electrochemical method script files, which can be used by an app executing on the smartphone to interact with a potentiostat chip, which can be located in the electronics layer 118 or optional secondary device 104. The electronics are able to time-stamp the beginning and end of sweat accumulation for each reservoir and monitor signals (e.g. current or voltage) from the electrochemical sensor which could be used for biosensors such as glucose and lactate.

Thus, an app 124 on a smartphone having a Bluetooth linked connection to the stackable structure 102 can measure and display the volume of collected sweat, calculate an instantaneous sweat rate, monitor the amperometric response to lactate, and other measurable parameters implemented by the biosensor patch 100. Moreover, the sweat collected into the sweat reservoirs can be later extracted and further analyzed with high collection efficiency and no cross-contamination.

In practical embodiments, the electronics 118 (and/or secondary device 104) can enable real-time feedback to monitor fatigue/stress, e.g., through a lactate biosensor. Moreover, the wearable biosensor patch 100 (e.g., wearable as an armband or adhesive patch) can feature a changeable sweat collection area, based on application.

### Second Example Modular Biosensor Patch

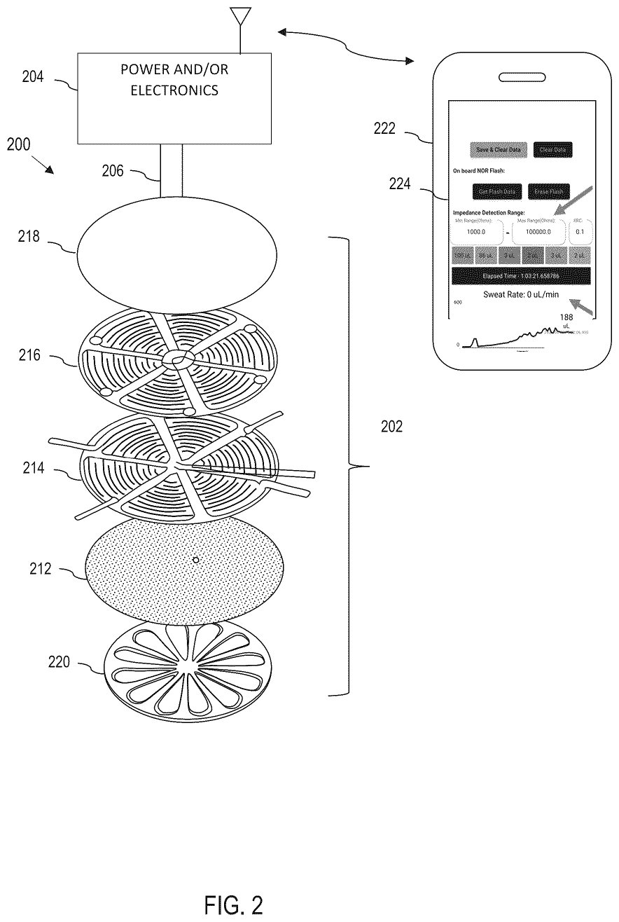

Referring now to FIG. 2, an example wearable biosensor patch 200 is illustrated, which enables sweat aggregation, analysis, and containment. The biosensor patch 200 includes some features that are analogous to those features described with reference to FIG. 1. As such, like structure is implemented with like reference numbers 100 higher in FIG. 2 compared to FIG. 1. In this regard, the description of FIG. 1 is incorporated by reference into the description of FIG. 2 except where expressly noted.

In some embodiments, the wearable biosensor patch 200 provides real-time human sweat monitoring. According to further aspects herein, the wearable biosensor patch 200 is able to collect and chronologically store sweat, e.g., in discrete reservoirs. The chronological collection of sweat enables applications in biomarker sensing and human performance monitoring analogous to that of FIG. 1.

In some embodiments, the biosensor patch 200 is implemented as a stackable structure 202. In other embodiments, the stackable structure 202 can connect to an optional secondary device 204 via suitable connection, e.g., a ribbon cable 206. The secondary device 204 can enclose battery power, optional additional electronics (e.g., processor, memory, transceiver, etc.), a combination thereof, etc. In this regard, the stackable structure 202 can define a disposable component, whereas the secondary device 204 can define a reusable component.

As illustrated, the wearable biosensor patch 200 is carried out using a multilayer stackable structure 202 including a medical grade adhesive layer 212, one or more laser induced graphene electrode layers 214, one or more Polydimethylsiloxane (PDMS) microfluidic layers 216, and an optional flexible electronic layer 218, in a manner analogous to that described with reference to FIG. 1.

Additionally, the stackable structure 202 adds an optional sweat wicking layer 220 under the medical grade adhesive layer 212. The sweat wicking layer 220 can improve the performance of sweat transfer from the individual to the inlet of the biosensor patch 200.

As noted in greater detail above, the stackable layers can be laminated together to create a disposable sweat patch. Also, features described with multiple layers can be consolidated into a single layer, e.g., by integrating LIG electrodes with microfluidics in a single layer, by integrating the electronics and LIG electrodes in a single layer, etc.

Moreover, the disposable components can connect to a reusable electronic board (e.g., within the electronics of the optional secondary device 204) that wireless communicates collected data, e.g., via Bluetooth to a remote processing device 222 (e.g., a smartphone). The smartphone can run an app 224 to display results of sweat collection and/or analysis as described more fully herein. In this regard, as the functionality and features are analogous to that described with reference to FIG. 1, repetitive details are omitted for compactness.

### Third Example Modular Biosensor Patch

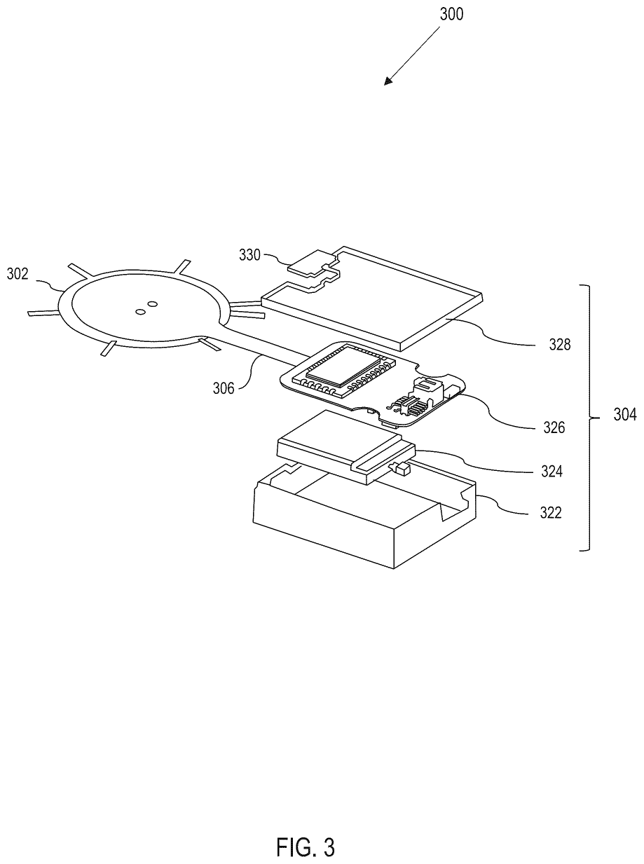

Referring to FIG. 3, yet another example wearable biosensor patch 300 is illustrated, which enables sweat aggregation, analysis, and containment. The biosensor patch 300 includes some features that are analogous to those features described with reference to FIG. 1 and/or FIG. 2. As such, like structure is implemented with like reference numbers 100 higher in FIG. 3 compared to FIGS. 2, and 200 higher in FIG. 3 compared to FIG. 1. In this regard, the description of FIG. 1 and FIG. 2 are incorporated by reference into the description of FIG. 3 except where expressly noted. Moreover, since aspects of communication with a remote processing device (e.g., a smartphone) running an app are analogous to that described more fully herein, the remote processing device is omitted for clarity and brevity.

In some embodiments, the wearable biosensor patch 300 provides real-time human sweat monitoring. According to further aspects herein, the wearable biosensor patch 300 is able to collect and chronologically store sweat, e.g., in discrete reservoirs. The chronological collection of sweat enables applications in biomarker sensing and human performance monitoring in a manner analogous to that of FIG. 1 and FIG. 2.

In some embodiments, the biosensor patch 300 is implemented as a stackable structure 302. In other embodiments, the stackable structure 302 can connect to an optional secondary device 304 via a ribbon cable 306. The secondary device 304 can enclose battery power, optional additional electronics, a combination thereof, etc. In this regard, the stackable structure 302 can define a disposable component, whereas the secondary device 304 can define a reusable component.

Analogous to that of FIG. 1 and FIG. 2, the wearable biosensor patch 300 is carried out using a multilayer stackable structure 302. Analogous to that described above with reference to FIG. 1 and FIG. 2, example layers can include a medical grade adhesive layer, one or more laser induced graphene electrode layers, one or more Polydimethylsiloxane (PDMS) microfluidic layers, a flexible electronic layer, and an optional sweat wicking layer in a manner analogous to that described with reference to FIG. 1 and FIG. 2. For sake of compact disclosure, the details of the individual layers are not duplicated.

The embodiment of FIG. 3 illustrates example detail for the optional device 304, which is illustrated as having a bottom case 322, a battery 324, additional electronics 326 within the bottom case 322, and a top lid 328 that slides closed over the top of the bottom case 322. The top lid 328 includes a hinge 330 that sits over the flexible ribbon cable 306.

### Modular Configuration

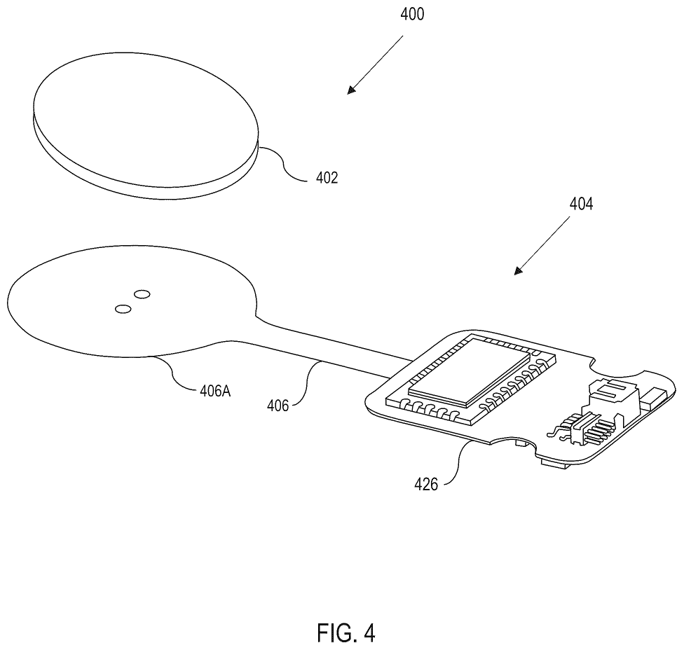

Referring to FIG. 4, as noted more fully herein, the various previous embodiments can be combined in any combination to derive a modular wearable biosensor patch 400, which enables sweat aggregation, analysis, and containment. In some embodiments, the wearable biosensor patch 400 provides real-time human sweat monitoring. According to further aspects herein, the wearable biosensor patch 400 is able to collect and chronologically store sweat, e.g., in discrete reservoirs. The chronological collection of sweat enables applications in biomarker sensing and human performance monitoring in a manner analogous to that of FIG. 1-FIG. 3. As such, analogous elements are illustrated with reference numbers 100 higher in FIG. 4 compared to FIG. 3, 200 higher in FIG. 4 compared to FIGS. 2, and 300 higher in FIG. 4 compared to FIG. 1. Thus, all aspects of FIG. 1-FIG. 3 are incorporated into FIG. 4 unless otherwise indicated. For instance, analogous to that of FIG. 3, since aspects of communication with a remote processing device (e.g., a smartphone) running an app are analogous to that described more fully herein, the remote processing device is omitted for clarity and brevity.

The illustrated modular biosensor patch 400 is implemented as a stackable structure 402 coupled to a device 404, e.g., via a flexible ribbon cable 406. The device 404 includes electronics 426, which can include a processor, memory, transceiver, etc.). Analogous to that of FIG. 1-FIG. 3, the wearable biosensor patch 400 is carried out using a multilayer stackable structure 402, which can include any combination of a medical grade adhesive layer, one or more electrode layers, one or more microfluidic layers, a flexible electronic layer, an optional sweat wicking layer, or any combination thereof, in a manner analogous to that described with reference to FIG. 1-FIG. 3. For sake of compact disclosure, the details of the individual layers are not duplicated.

The embodiment of FIG. 4 illustrates an example modular sensor. In some embodiments, the stackable structure 402 and optionally, the flexible ribbon cable 406, form the disposable component, whereas the device 404 (e.g., electronics, battery, etc.) form a reusable component. In other embodiments, the stackable structure 402 forms the disposable component, whereas the device 404 and flexible ribbon cable 406 form the reusable component. In this regard, the embodiment of FIG. 4 is largely analogous to the embodiment of FIG. 3. However, a case around the electronics 426 is omitted for clarity of illustration. In practice, the electronics 426 can be housed in a suitable enclosure, which may also house a suitable battery to power the electronics. For instance, a case and battery analogous to the bottom case 322, battery 324, and top lid 328 of FIG. 3 can be implemented.

FIG. 4 also illustrates a generally circular feature 406A of the flexible ribbon cable 406 that interfaces with the electrodes and optionally other features of the stackable structure 402. In this regard, the feature 406A of the flexible ribbon cable 406 can couple to the stackable structure 402, e.g., by coupling to a top layer, by coupling to a bottom layer, or by sandwiching between any adjacent layers of the stackable structure 402.

### Microfluidic Device Configuration

According to aspects herein, a microfluidic device is provided for collecting liquid samples. Referring to FIG. 5, an example microfluidic device 500 is illustrated according to aspects of the present disclosure. The microfluidic device 500 can be used in the PDMS layer of any of the previous implementations described with reference to FIG. 1-FIG. 4 herein.

The illustrated microfluidic device includes several storage reservoirs 502. In this regard, there are six storage reservoirs illustrated, including a first reservoir 502A, a second reservoir 502B, a third reservoir 502C, a fourth reservoir 502D, a fifth reservoir 502E, and a sixth reservoir 502F. In order to chronologically store sweat and facilitate reliable, complete, and air-free filling, the illustrated microfluidic device implements each storage reservoir in a serpentine pattern 504. In the illustrated design, six discrete reservoirs 502 are connected such that the reservoirs 502 fill sequentially, with each successive reservoir 502 only beginning to fill after no more sweat could be added to the previous reservoir 502. In this regard, the previous reservoir 502 need not fill to its fullest capacity. Rather, the previous reservoir 502 fills until it no longer accepts additional fluid, upon which, the next reservoir 502 begins to fill.

For example, a liquid such as sweat enters an inlet 506. The inlet 506 directs the liquid to an input of the serpentine pattern 504 of the first reservoir 502A, thus the first reservoir 502A fills first. The first reservoir 502A closes, then, the serpentine pattern 504 of the second reservoir 502B fills. The second reservoir 502B closes, then, the serpentine pattern 504 of the third reservoir 502C fills. The third reservoir 502C closes, then, the serpentine pattern 504 of the fourth reservoir 502D fills. The fourth reservoir 502D closes, then the serpentine pattern 504 of the fifth reservoir 502E fills. The fifth reservoir 502E closes, then the serpentine pattern 504 of the sixth reservoir 502F fills. Once the sixth reservoir 502F fills, that reservoir is closed. That is, the sequential pattern repeats until the serpentine pattern 504 of each reservoir 502 is filled. In some implementations, after filling all of the reservoirs 502, the microfluidic directs the liquid to an outlet 507 of the sweat patch for overfilling.

In the example implemented with regard to FIG. 5, the reservoirs fill completely in a sequential, yet independent manner. That is, fluid flows through the serpentine of the first reservoir 502A without entering any other reservoirs 502B-502F. Once the first reservoir 502A is full, fluid is redirected into the second reservoir 502B. Here, the new fluid does not flow through the first reservoir 502A. Rather, the new fluid flows directly into the second reservoir 502B, bypassing the first reservoir 502A. The fluid in the first reservoir 502A remains undisturbed. Once the second reservoir 502B is filled, the new fluid flows directly into the third reservoir 502C, etc. The process repeats until the entire microfluidic device is full. This prevents contamination of an already filled reservoir.

As will be described more fully herein, valves can be utilized to control the flow of liquid through the reservoirs.

In some embodiments, soft lithography casting techniques can be used to produce PDMS microfluidic devices, which exhibit biocompatibility, flexibility, chemical stability, and transparency. Moreover, PDMS facilitates configuring crosslinking ratios and curing times to tailor the elasticity of the PDMS microfluidic. For this microfluidic application, the PDMS needs to be flexible enough to conform to an individual for a wearable application and elastic enough to be easily peeled off the mold without tearing. Additionally, if the PDMS is not cross-linked enough, the microfluidic channels and the reservoir geometry will change while bending, causing inconsistencies in fluid flow and different amounts of sweat that is stored, respectively. These changes can cause large inconsistences in the sweat rate sensor as it quantifies the fluid flow based on the geometry of the reservoir.

Reservoir volume can be increased by reducing the unused space on the microfluidic device, including reducing space between reservoirs 502 as well as reducing wall thickness within the reservoirs 502. In an example implementation, a reservoir height of 500 μm resulted in a theoretical reservoir volume of 94 μL and a total sweat storage volume of 564 μL.

Continuing with FIG. 5, the reservoirs 502 can integrate a sweat rate sensor that can be utilized to monitor sweat collection and to timestamp sample collection. For instance, the sweat rate sensor can be implemented as a laser induced graphene electrode implementing a serpentine pattern through the microfluidic. That is, a pair of electrodes can follow the serpentine pattern of each reservoir (e.g., so as to not impede the flow of sweat into the reservoir). When the electrodes are coupled to suitable electronics, flow rate can be measured, as described more fully herein.

Moreover, each reservoir 502 can include a sample port 512 that can be utilized to extract fluid from an associated reservoir 502.

### Capillary Burst Valve

As noted previously, capillary burst valves 508 (CBV) can be utilized to control the filling of the reservoirs 502. In FIG. 5, each capillary burst valve 508 is schematically represented by the small triangle. In an example embodiment, the capillary burst valves utilize the user's perspiration pressure as the driving force in the microfluidic. These approaches offer the benefit of not requiring external support equipment to drive flow. For instance, capillary burst valves can be positioned after an inlet of each reservoir to control sweat progress and ensure sequential filling into the reservoirs. These valves provide passive control of flow by introducing a sudden expansion in the channel, which confines the meniscus at the entrance of the expansion until the driving force can overcome the increased pressure barrier. This pressure is overcome when the previous reservoir is full and the increased driving force bursts the valve.

Referring to FIG. 6, an example schematic diagram 600 illustrates a partial path of a reservoir of a microfluidic device, showing an arrangement of capillary burst valves 602, 604 that can control reservoir filling.

In some embodiments, the bursting pressure (BP) of a capillary burst valve can be controlled via the valve geometry. As an example, burst pressure can be described by:

$$B P = - 2 σ [ \frac{cos ( {θ}_{s} )}{w} + \frac{cos ( {θ}_{v} )}{h} ]$$

where w and h are the width and height of the channel, respectively, σ is the fluid surface tension, and θₛ and θᵥ are the advancing angle with the sidewall and with the top and bottom of the channel, respectively.

The capillary burst valves 602 burst upon complete filling of the previous reservoir while resisting the increased driving force from higher flow rates.

Capillary burst valves 604 can also be incorporated at the end of each reservoir to prevent liquid (e.g. sweat) exiting, e.g., through an air vent (which can be utilized for extraction of the sample from the reservoir at the time of sample analysis). The geometry of these capillary burst valves can be designed to require a higher bursting pressure than sequential flow capillary burst valves, providing a higher impedance to flow and driving flow to the next reservoir.

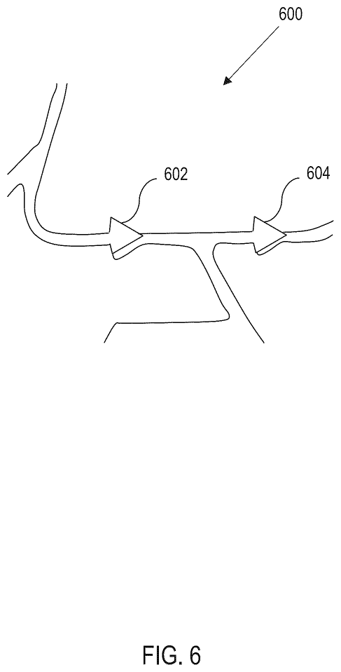

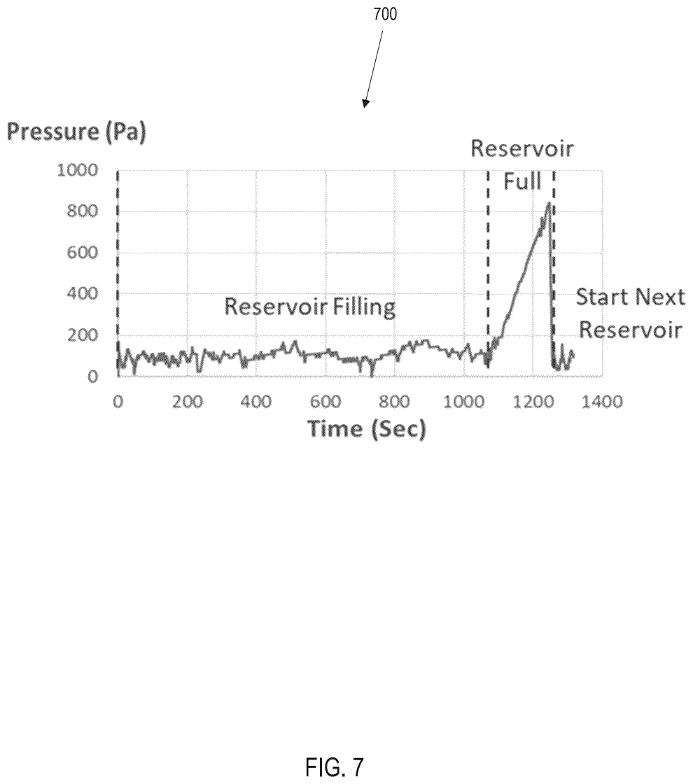

Referring to FIG. 7, a plot 700 illustrates internal pressure of microfluidic depicting different stages of filling a reservoir. Under slow flowing rates, the internal pressure of the microfluidic is relatively flat while the reservoir is filling. The internal pressure increases until it is greater than the bursting pressure of an associated capillary burst valve, and the next reservoir begins to fill. Collectively, capillary burst valves can successfully control the sweat flow via passive Laplace pressure, resulting in complete and sequential filling of the microfluidic.

### Laser Induced Graphene Electrodes

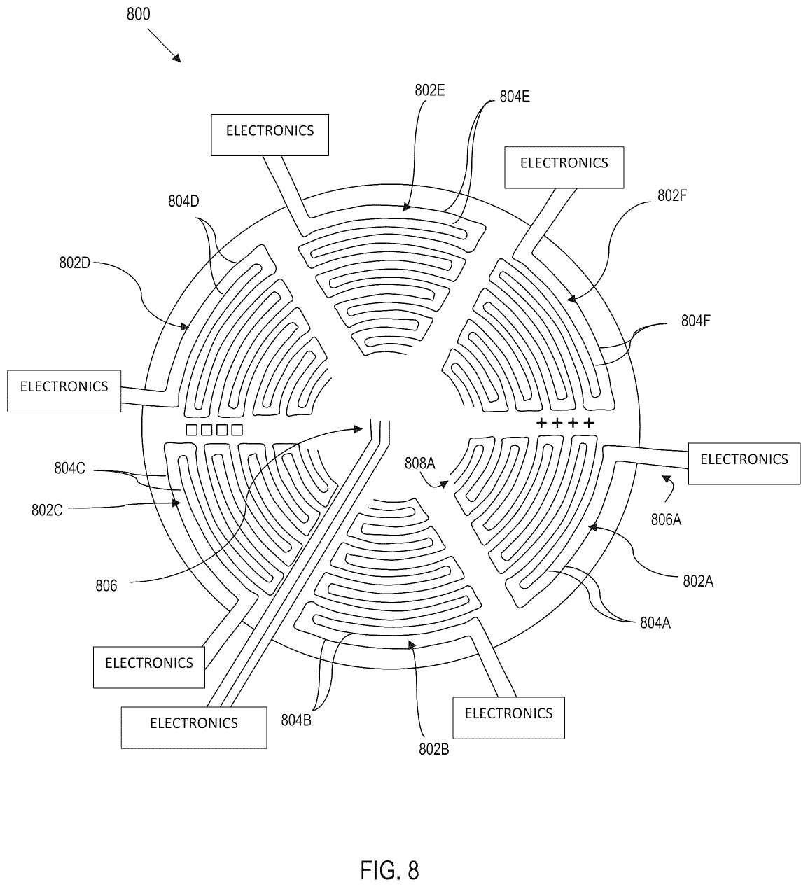

Referring to FIG. 8 in some embodiments, a microfluidic device 800 includes one or more reservoirs, e.g., reservoirs 802A-802F in this example implementation. In this regard, a set of parallel band electrodes 804A-804F can be provided for passive sweat rate sensing. As illustrated, the parallel band electrodes 804A-804F generally follow the geometry of the reservoirs 802A-802F.

In an example embodiment, the parallel electrodes are implemented by parallel serpentine electrodes that weave through the channels of each reservoir. Laser induced graphene is utilized to fabricate the parallel electrodes, providing a high sensitivity, ability to tailor the resistance of the electrodes, and allowed for rapid fabrication.

Notably, the LIG electrodes do not impede sweat flow through the microfluidic channels. To operate the sweat rate sensor, an electrochemical signal is applied to the parallel electrodes of the LIG which weave through the serpentine microfluidic channels that form the reservoirs.

For instance, an electrochemical signal can be applied across the pair of electrodes 804A to measure a condition associated with the reservoir 802A. By way of example, based on a detected change in a real (series) resistance (or impedance), the volumetric flow rate of the sweat is measured.

Analogously, an electrochemical signal can be applied across the pair of electrodes 804B to measure a condition associated with the reservoir 802B; an electrochemical signal can be applied across the pair of electrodes 804C to measure a condition associated with the reservoir 802C; an electrochemical signal can be applied across the pair of electrodes 804D to measure a condition associated with the reservoir 802D; an electrochemical signal can be applied across the pair of electrodes 804E to measure a condition associated with the reservoir 802E; and an electrochemical signal can be applied across the pair of electrodes 804F to measure a condition associated with the reservoir 802F.

In an example implementation, initially the electrodes have infinite resistance. For instance, the electrochemical signal can be applied across the electrodes 804A at a first end 806A thereof. In this example, the distal end of the parallel electrodes 804A are floating, creating an open circuit. However, when a fluid such as sweat enters the channel, the fluid shorts the leads of the electrode pair 804A. Further filling the reservoir with sweat causes the series resistance of the parallel electrodes to decrease.

A calibration curve can be constructed, or other suitable techniques can be utilized to convert a change of resistance into a flow measurement. For instance, a normalization algorithm can be utilized to factor out differences in LIG resistances across electrodes.

In some implementations, an electrical device may not be able to read an open circuit condition. In this regard, the ends 808A of the electrode pair 804A can be tied together, tied to a load, or otherwise reconfigured in order to enable the suitable electronics to function. For instance, the laser induced graphene electrodes can form a closed loop, which sets the initial resistance to a relatively high value. Such an approach may be beneficial, depending upon the detection electronics/circuitry utilized by the device. Additionally, with a closed loop, a fixed resistance can be measured before sweat has entered the reservoir. By measuring this resistance during fabrication, proper fabrication of the sweat sensor within the patch can be ensured.

The device 800 can also include a sensor, such as a multi-electrode electrochemical sensor 806. This sensor 806 is illustrated having three electrodes, e.g., a working electrode, a reference electrode, and a counter electrode. Examples of a three electrode sensor are described more fully herein.

Thus, as illustrated in FIG. 8, LIG electrodes for the sweat patch can include two parallel electrodes for each reservoir (thus six pairs of electrodes in this example). The parallel electrodes can be utilized for the sweat rate sensing of the associated reservoir as it fills with sweat. The sweat patch can also include one or more 3 electrode sensor for electrochemical biosensing.

To construct the flexible electrode design (e.g., to construct the parallel band electrodes), a laser engraver can be used to fabricate laser induced graphene electrodes on polyimide film. The high-power laser is able to rapidly carbonize the polyimide into conductive carbon electrodes. During carbonization, the sp³ carbon atoms are photothermally converted to sp² (graphite/graphene-like planar geometry).

Laser settings can be optimized, for instance, by changing laser power, raster rate, etc., in order to minimize electrode resistance, form a smooth and uniform texture, and minimize warping of the polyimide film.

In some embodiments, the performance of the LIG surface provides an ability to continue to function after prolonged exposure to sweat, e.g., in excess of 24 hours. In practical applications, the system should prevent adsorption of biological agents onto the laser-induced graphene electrodes.

Reducing adsorption retains the concentration of biological materials within the sweat and preserves the fidelity of sweat samples stored in the patch. For instance, oxygen plasma treatment favorably increases the hydrophilicity of the LIG, thus decreasing adsorption by increasing the energy barrier for proteins to displace water molecules at the surface. Additionally, a nitric acid treatment can further oxidize the polyimide and LIG.

Droplets of water on an untreated LIG may maintain their semispherical shape) (>90°. By comparison, droplets of water on a plasma treated LIG were immediately wicked along the lines, demonstrating an increased hydrophilicity. This indicates that the surface hydrophilicity was increased, confirming that oxygen plasma treatment is a suitable for reducing biological adsorption.

Within the PDMS-polyimide device integrating serpentine LIG electrodes into the microfluidic device, complete filling may be influenced by the degree of hydrophilicity of the graphene electrodes lining each reservoir. For instance, the LIG may act as a barrier to sweat flow due to its hydrophobicity and raised geometry, essentially creating separate lanes within the channels, preventing complete filling. In order to reduce this barrier, the hydrophilicity of the LIG can be tailored, e.g., via treatment with oxygen plasma and/or nitric acid.

In this regard, the hydrophilicity of the LIG electrodes can be improved via treatment with nitric acid. For instance, treatment with dilute nitric acid can successfully improve channel filling while retaining the LIG's conductivity. However, higher concentrations of acid or prolonged exposure increased the hydrophilicity too much, leading to wicking along the edge of the channels ahead of the sweat front and premature bursting of the capillary burst valves.

### Integration of a Biomarker Sensor

As another example, three parallel electrodes 806 (working, reference, and counter), can be created for the electrochemical biosensor.

A biomarker sensor, e.g., a lactate biosensor, can be integrated into the sweat patch microfluidic structure, e.g., into the electrode layer 114, FIG. 2, electrode layer 214, FIG. 2, etc.

Lactate is convenient due to its presence in sweat and because of a correlation between concentration and activity level. Unfortunately, conventional lactate biosensors saturate too easily, typically well below the physiologically relevant concentration required for the present application (up to 100 mM).

However, in another example implementation, a biosensor (biomarker sensor) can comprise three LIG electrodes, including a working electrode, a counter electrode, and a reference electrode. A catalyst, such as platinum, can be deposited on working and counter electrodes. Moreover, in some embodiments, the working electrode can be platinized, functionalized with an amine binding chemistry (e.g., EDC/NHS), and covalently bonded to Lactate Oxidase, which functions as a biorecognition element for a biosensor.

Here, EDC/NHS binds the lactate Oxidase to the surface of the graphene. The lactate oxidase breaks down lactate into pyruvate and hydrogen peroxide. Hydrogen peroxide can then by broken down by the catalyst (e.g., platinum), generating a detectable electrical signal. Moreover, Ag/AgCl can be screen printed and cured on the reference electrode. In some embodiments, an oxygen permeable membrane may be utilized to hinder the diffusion of lactate.

Aspects herein provide a working electrode that has been platinized, functionalized with EDC/NHS, and covalently bonded with Lactate Oxidase, a platinized counter electrode, a screen-printed Ag/AgCl reference electrode, or combinations thereof. As such, aspects herein construct sensors that integrate Ag/AgCl reference electrode and platinum counter electrode with the LIG electrode design. Three LIG electrodes can be fabricated in parallel. Moreover, the platinum can be electrodeposited on the working and counter electrodes, while Ag/AgCl is screen printed and cured on the reference electrode.

To increase the sensitivity of the LIG electrode, platinum nanoparticles can be electrodeposited on the surface of the LIG by applying a very high current density to the surface of LIG in chloroplatinic acid for a very short time interval, which results in nucleated nanoparticles. Simple amine binding chemistry (EDC/NHS) can be utilized to bind lactate oxidase (LO) to the surface of the graphene working electrode. The linear sensing range of the biosensor can be configured by decreasing the amount of enzyme functionalized to the sensor surface and adding blocking the surface with bovine serum albumin (BSA).

In order to shift the reaction away from oxygen limited conditions, oxygen permeable membranes may be used to hinder the diffusion of lactate. For instance, Nafion, glutaraldehyde (GA), and chitosan can be utilized as an oxygen permeable membrane. Using 1% glutaraldehyde with 10 mg/mL BSA can provide a linear sensing range between 0-100 mM, which is ideal for sensing lactate in sweat.

### Cross-Contamination

In order to maintain the purity of the collected sweat samples at different time-stamped intervals, diffusion and mixing across reservoirs should be minimized. For instance, convective mixing during vigorous movement such as aerobic activity is a potential source of sample cross-contamination.

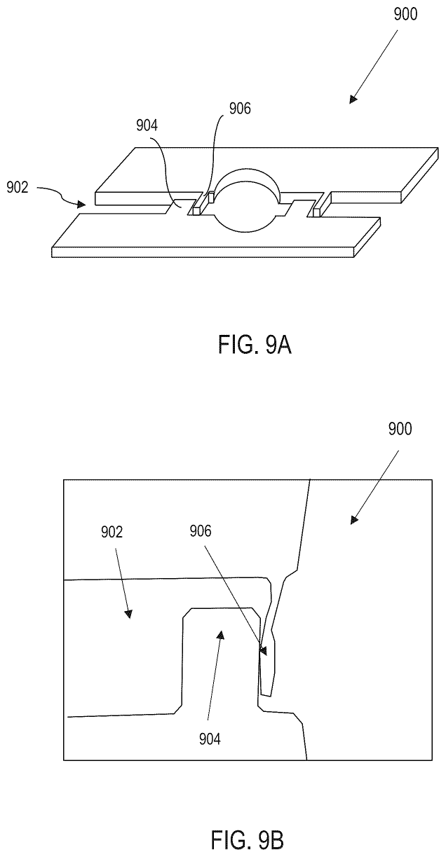

Referring to FIGS. 9A and 9B generally, a mechanical check valve 900 can be used to prevent fluid backflow out of the reservoirs. The illustrated check valve 900 includes a channel 902, a stopper 904 and a flap 906. The flap 906 serves as a one-way gate to prevent reverse flow of a liquid through the check valve 900 back into the channel 902. That is, fluid can flow only from the channel 902, through a passage adjacent to the stopper, and around the flap. However, as best illustrated in FIG. 9B, a reverse direction pushes the flap 906 against the stop 904, preventing reverse fluid flow.

As an alternative to check valves, microfluidic rectifiers can allow fluid to flow more easily in one direction than another.

Mechanical check valves and microfluidic rectifiers increase design and manufacturing complexity. Moreover, mechanical check valves and microfluidic rectifiers may fail to prevent contamination from natural diffusion.

### First Example Hydrogel Plug

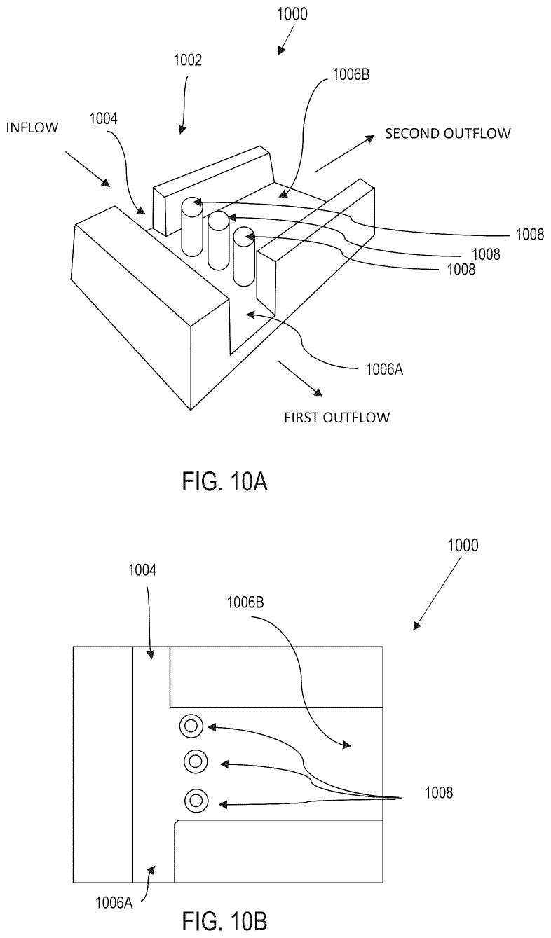

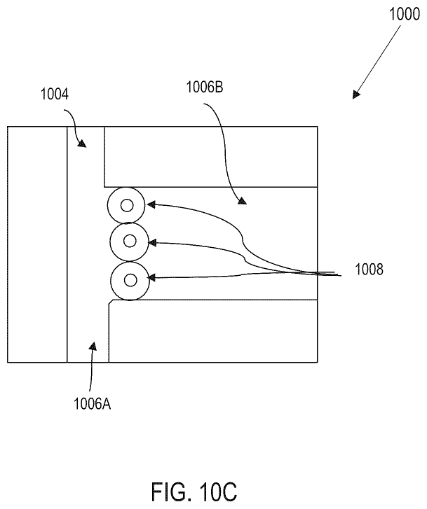

Referring to FIG. 10A, 10B, and 10C generally, a valve incorporating a moisture responsive polymer can prevent backflow, contamination, or both from natural diffusion. As an example, a valve can be constructed that utilizes moisture expanding polymers designed to swell and plug a channel. As an example application, a hydrogel polymer valve can be used for closing off an inlet of a reservoir (e.g., reservoirs 502A-502F, in the microfluidic device 500, FIG. 5). Notably, if an inlet of a reservoir is open, then sweat can be pulled out of the inlet when a different reservoir is being extracted, causing cross-contamination. However, the hydrogel Plug can prevent such cross-contamination.

Referring to FIG. 10A, a microfluidic device 1000 includes a channel system 1002 that includes a first channel path defined by an inflow 1004 and a first outflow 1006A.

The channel system 1002 also includes a second channel path defined by a second outflow 1006B.

One or more (three as illustrated) hydrogels 1008 are positioned so as to traverse across the second outflow 1006B. As illustrated, the hydrogels 1008 laterally span across the second channel path orthogonal to the flow direction of the second outflow 1006. In the illustrated implementation, the hydrogels 1008 are further positioned adjacent to the first channel path (adjacent to the inflow 1004 and the first outflow 1006A). However, in practice, the hydrogels 1008 can alternatively be positioned inset into the second outflow 1006B.

FIG. 10B is a top view illustrating the microfluidic device 1000 of FIG. 10A, where the hydrogels 1008 are in an expanding state. In the illustrated state, fluid flowing into the inflow 1004 can flow into the first outflow 1006A and the second outflow 1006B.

FIG. 10C is a top view illustrating the microfluidic device 1000 of FIG. 10A, where the hydrogels 1008 are in a fully expanded state. In the illustrated state, fluid flowing into the inflow 1004 can flow into the first outflow 1006A. However, fluid flowing into the inflow 1004 cannot flow into the second outflow 1006B. Here, the hydrogels 1008 have swollen and formed plugs that collectively block fluid from flowing along the second outflow 1008B.

### Second Example Hydrogel Plug

Various hydrogel valve configurations can be implemented using moisture expanding polymers.

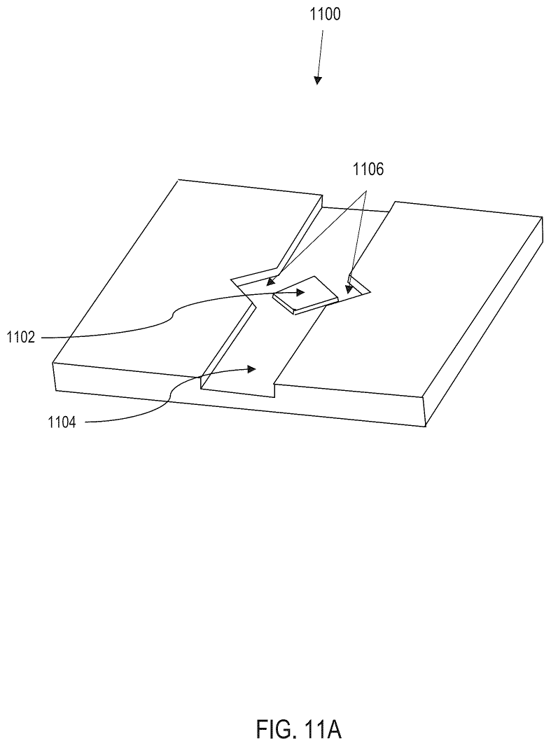

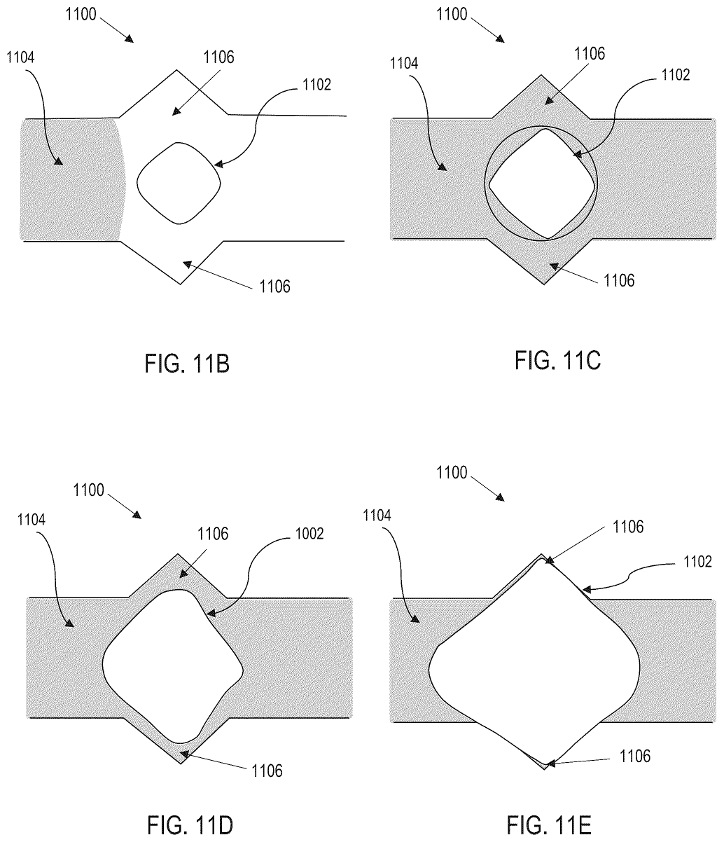

For instance, referring to FIG. 11A, a microfluidic device 1100 is illustrated. The microfluidic device 1100 includes a moisture expanding polymer that is utilized to construct a valve 1102. The valve 1102 is seated in a microfluidic channel 1104 so as to leave a pair of bypass pathways 1106 within the microfluidic channel 1104 adjacent to either side of the valve 1102.

For instance, the valve 1102 may comprise a photopolymerized sodium acrylate hydrogel that is patterned in the microfluidic channel 1104. The hydrogel valve 1102 swells when in contact with water/sweat. Based upon the design of the valve 1102 and the corresponding microfluidic channel 1104, the valve 1102 can be utilized to close off the microfluidic channel 1104.

Referring to FIG. 11B, the valve 1102 of the microfluidic device 1100 is about to be contacted by a fluid, e.g., sweat. Here, the sweat can travel along the microfluidic channel 1104 including the bypass pathways 1106.

Referring to FIG. 11C, the valve 1102 of the microfluidic device 1100 is in contact with sweat. As such, the valve 1102 begins to swell. Here, the sweat can still travel along the microfluidic channel 1104 including the bypass pathways 1106.

Referring to FIG. 11D, the valve 1102 of the microfluidic device 1100 remains in contact with the sweat and thus the valve 1102 continues to swell. Here, the sweat can still travel along the microfluidic channel 1104, but the bypass pathways 1106 are starting to become constricted.

Referring to FIG. 11E, the valve 1102 of the microfluidic device 1100 has swollen to the point that the bypass pathways 1106 are closed off. Here, the sweat in the microfluidic channel 1104 to the left of the valve 1102 is isolated from the sweat in the microfluidic channel 1104 to the right of the valve 1102.

### Example Slow Hydrogel Valve

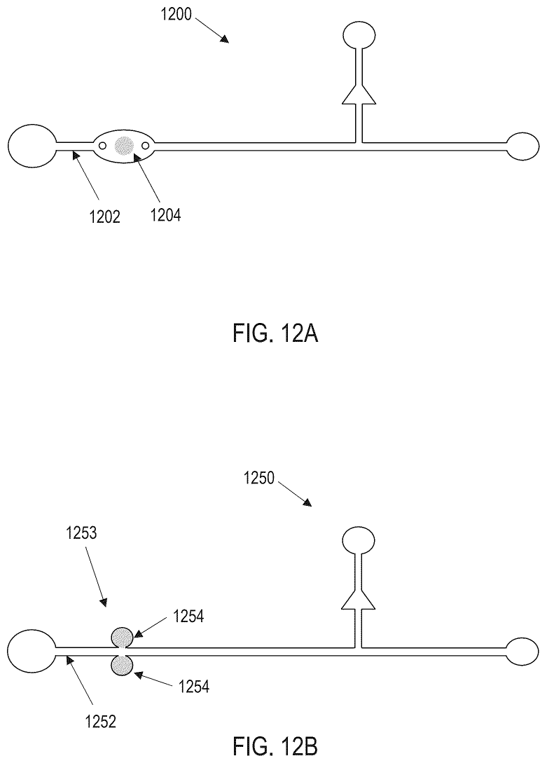

Referring to FIGS. 12A and 12B, example “slow” hydrogel valves are illustrated.

Referring particularly to FIG. 12A, a microfluidic device 1200 includes a microfluidic channel 1202. In-line with the microfluidic channel 1202 is a hydrogel valve 1204. In the example implementation illustrated, a central hydrogel valve 1204 may be utilized. Configuring the hydrogel valve 1204 to be slow acting (e.g., 1-2 hour rate from time of exposure to sweat to closing), allows the microfluidic channel 1202 to fill prior to closing of the hydrogel valve 1204.

This approach is relatively simple to make, but exhibits low resolution as the hydrogel valve 1204 will not be confined to a specific location.

Referring to FIG. 12B, another example configuration of a “slow” valve system is illustrated. A microfluidic device 1250 includes a microfluidic channel 1252. A hydrogel valve system 1253 is positioned along the microfluidic channel 1252. In the example implementation illustrated, the hydrogel valve system 1253 includes two or more hydrogel valves 1254 aligned circumferentially at a designated location along the microfluidic channel 1252.

The approach illustrated in FIG. 12B is relatively more complex to assemble compared to the central hydrogel valve 1204 of FIG. 12A. However, the configuration of FIG. 12B decreases the distance each hydrogel valve 1254 of the hydrogel system 1253 has to swell. Confining each hydrogel valve 1254 to a small well and positioning the hydrogel valves 1254 circumferentially on either size of the microfluidic channel 1252 increases reproducibility. Additionally, as only a small amount of liquid (e.g., sweat) will come in contact with each hydrogel valve 1254, the swelling rate can be limited, increasing the time it takes for the valve system 1253 to close. This approach also holds the hydrogel valves 1254 in place while it swells until there is enough tension for them to burst from the well, collapsing the microfluidic channel 1252.

As with the example of FIG. 12A, configuring the hydrogel valves 1254 to be slow acting (e.g., 1-2 hour rate from time of exposure to sweat to closing), allows the microfluidic channel 1252 to fill prior to closing of the hydrogel valves 1254.

In another example implementation, hydrogels can be provided on either side of the microfluidic.

Referring to FIG. 12A and FIG. 12B generally, in an example implementation, a custom slow swelling hydrogel valve (e.g., valve 1204, FIG. 12A; valve 1254, FIG. 12B) is composed of acrylic acid, a crosslinker, and photoinitiator. Such a hydrogel valve may exhibit a significant volume expansion (e.g., a 4x volume expansion) over a multi-hour time period (e.g., 2 hour) when placed in water/sweat. Moreover, the slow swelling hydrogel valve(s) can immediately close off a corresponding inlet after an associated reservoir is completely filled. In an example configuration, the hydrogel valve(s) can isolate a channel and direct flow to another channel by increasing the pressure in the channel. As the hydrogels absorb water and swell, a corresponding flow channel is closed (e.g., in approximately 1 hour), increasing the pressure in the channel enough to burst the capillary burst valve and exit another outlet.

### Example Fast Closing Hydrogel Valve

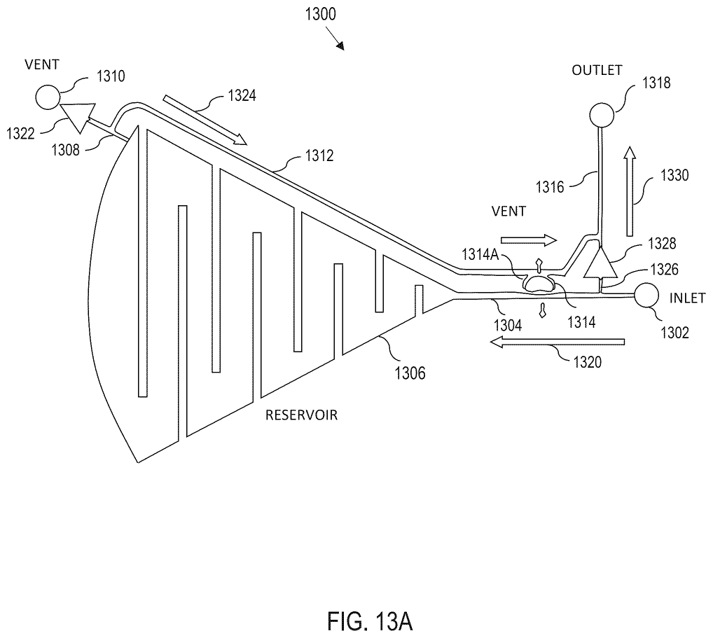

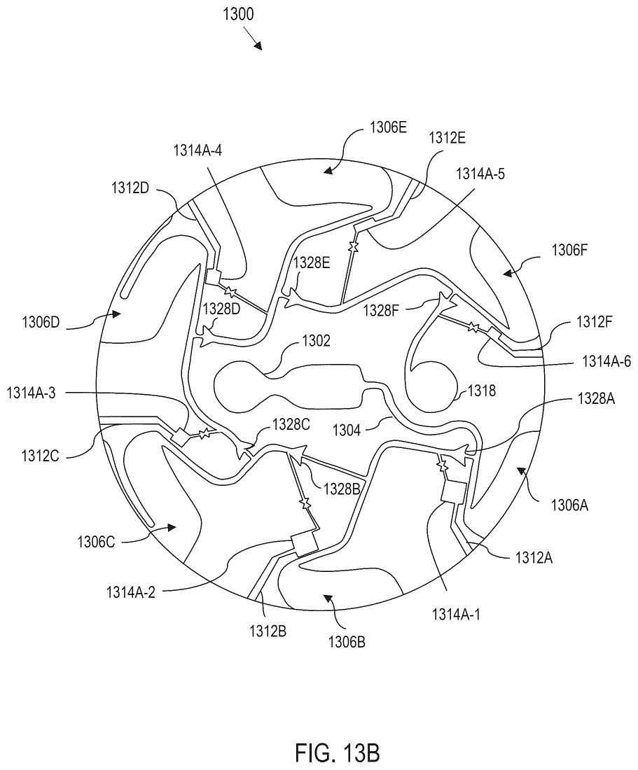

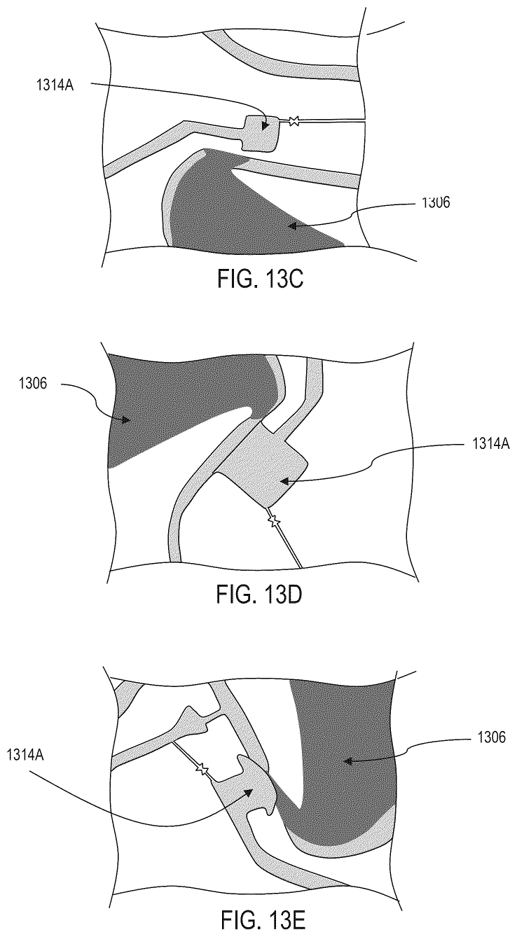

A fast-closing hydrogel valve can be realized utilizing a fast-swelling hydrogel. Referring to FIG. 13, a microfluidic system 1300 includes generally, an inlet 1302, an inlet channel 1304, and a serpentine reservoir 1306 that is coupled to the inlet 1302 by the inlet channel 1304. The microfluidic system also comprises an output channel 1308 coupled to an end section of the reservoir 1306. The output channel 1308 forks to a vent 1310, and a return channel 1312. The return channel 1312 in the illustrated example, extends back towards the inlet 1302 along a side of the reservoir 1306, then extends generally parallel to the inlet channel 1304. A hydrogel valve 1314 seats in the return channel 1312 (e.g., in a hydrogel valve chamber 1314A) along the section running generally parallel to the inlet channel 1304, e.g., proximate to an entrance to the reservoir 1306. The return channel 1312 then feeds into an outlet channel 1316 which leads to an outlet 1318.

In operation, sweat flows in the inlet 1302, then travels along the input channel 1304 to the reservoir 1306 in the direction of arrow 1320. When the reservoir 1306 is completely filled, sweat, sweat flows into the output channel 1308. A valve 1322, e.g., a capillary burst valve, prevents the sweat from flowing to the vent 1310. Rather, the vent can be utilized to extract the sweat from the reservoir 1306 at a time where analysis of the sweat is to be performed. As such, the fork in the output channel 1308 allows the sweat to flow into the return channel 1312, along the direction of arrow 1324.

As the sweat flows along the return channel 1324 in the area parallel to the input channel 1304, the sweat comes in contact with the hydrogel valve 1314. The corresponding hydrogel valve chamber 1314A is separated from the inlet of the reservoir 1306 and/or a wall of the inlet channel 1304 by a thin wall of PDMS. The hydrogel valve 1314 will then swell very quickly (e.g., within minutes). The swelling pressure applied to the wall of the PDMS forces the wall to deform and close off both the inlet to the reservoir 1306 and a side channel 1326 leading to a valve 1328 (e.g., a capillary burst valve).

In some implementations, the hydrogel valve 1314 comprises a

photopolymerized sodium acrylate hydrogel that can be patterned in a microfluidic channel, e.g., the hydrogel chamber 1314A. As noted above, the swelling can close off the channel in minutes, such as within 1 to 30 minutes, and in some examples, around 5-20 minutes. However, the close rate should be tuned such that the channel fills prior to closing.

After the valve 1328 closes, the internal pressure of the microfluidic will increase causing the valve 1328 (e.g., a capillary burst valve) to burst, allowing for the sweat to flow into the outlet channel 1316 to the outlet 1318 (e.g., in the direction of arrow 1330). This sweat can then be directed, for example, to fill a next reservoir in series.

This reduces the risk of reservoirs filling in parallel and conserves the chronological character of the samples stored in each reservoir.

Moreover, the hydrogel valve 1314 is not only useful for preventing contamination between reservoirs, but is also useful for sweat extraction via the vent 1310. If the hydrogel valve 1314 does not close off the inlet 1302 to the reservoir 1306, sweat can be pulled from one reservoir into the next when extracted (e.g., instead of venting through the outlet port 1318. Thus, the hydrogel valve 1314 further assists with reliable extraction of collected sweat samples.

Different geometries, wall thicknesses, cavity sizes, inlet channel widths, and swelling stress relief affect operation of the hydrogel valve. For instance, it may be easier to place hydrogel devices in larger cavities. Decreasing the wall thickness allows the hydrogel to flex easier, but if the wall thickness is too thin the hydrogel can burst through the wall and enter the collection reservoir. Decreasing the inlet channel width can increase the speed that the valve closes and can provide more effective isolation. However, decreasing the inlet channel width can increase the pressure drop into the reservoir, causing premature bursting of the capillary burst valves and skipping of the collection reservoirs.

In this regard, aspects herein provide a hammerhead swelling stress relief design. This “hammerhead shape” allowed the walls of the microfluidic to more easily protrude into the +inlet channel.

Due to the hammerhead shape of the hydrogel cavity, the side wall is able to consistently flex across the reservoir channel inlet. For instance, a hammerhead shape was constructed that flexed across a 200 μm inlet.

According to aspects herein, a combination of capillary burst valves and fast-swelling hydrogel valves direct fluid collection such that fluid (e.g., sweat) completely fills a set of reservoirs in chronological order, even with extremely high flow rates, e.g., on the order of 40 L/minutes. Thus, an example patch with six reservoirs as described more fully herein, may fill entirely in 15 minutes, thus much faster than a person can sweat. Moreover, the microfluidic properly flows the sweat into the hydrogel channel. For instance, after approximately 10 min, the hydrogel valves can completely swell, closing the inlets to the microfluidics, effectively isolating the reservoirs. In some configurations, the hammer head shape of the hydrogel channel allows the wall to deform easily, improving the reliability of the system.

Referring to FIG. 13B, another example microfluidics system 1300 is illustrated. This microfluidics system 1300 is analogous to the microfluidics system 1300 of FIG. 13A unless otherwise noted. As such, the description of FIG. 13A is adopted by reference into the description of FIG. 13B.

Whereas FIG. 13A illustrates a detailed example of a single reservoir system, FIG. 13B shows example components of microfluidic routing for an entire patch, which contains six reservoirs in this illustrative example. Because details of the reservoir, return channel, vent, etc., are illustrated in FIG. 13A, some of these features are omitted from FIG. 13B for clarity. Rather, in FIG. 13B, a portion of the microfluidics is illustrated to show the relationship of the hydrogel chambers for all six reservoirs to more clearly illustrate the sequential filing of the reservoirs.

Briefly, sweat flows into an inlet 1302. The sweat then follows an inlet channel 1304 that couples the inlet 1302 to a first reservoir 1306A. Upon filling the first reservoir 1306A, sweat flows along a return channel 1312A associated with the first reservoir 1306A to a first hydrogel valve chamber 1314A-1. A hydrogel valve within the hydrogel valve chamber 1314-1 (see description with regard to FIG. 13A) swells closing off the first reservoir 1306A. This causes a first capillary burst valve 1328A to rupture, allowing sweat entering the inlet to flow into a second reservoir 1306B. Notably, new sweat does not pass through the first reservoir at this time.

Analogous to that above, upon filling the second reservoir 1306B, sweat flows along a return channel 1312B associated with the second reservoir 1306B to a second hydrogel valve chamber 1314A-2. A hydrogel valve within the second hydrogel valve chamber 1314-2 swells closing off the second reservoir 1306B. This causes a second capillary burst valve 1328B to rupture, allowing sweat to flow into a third reservoir 1306C.

Analogous to that above, upon filling the third reservoir 1306C, sweat flows along a return channel 1312C associated with the third reservoir 1306C to a third hydrogel valve chamber 1314A-3. A hydrogel valve within the third hydrogel valve chamber 1314-3 swells closing off the third reservoir 1306C. This causes a third capillary burst valve 1328C to rupture, allowing sweat to flow into a fourth reservoir 1306D.

Analogous to that above, upon filling the fourth reservoir 1306D, sweat flows along a return channel 1312D associated with the fourth reservoir 1306D to a fourth hydrogel valve chamber 1314A-4. A hydrogel valve within the fourth hydrogel valve chamber 1314-4 swells closing off the fourth reservoir 1306D. This causes a fourth capillary burst valve 1328D to rupture, allowing sweat to flow into a fifth reservoir 1306E.

Analogous to that above, upon filling the fifth reservoir 1306E, sweat flows along a return channel 1312E associated with the fifth reservoir 1306E to a fifth hydrogel valve chamber 1314A-5. A hydrogel valve within the fifth hydrogel valve chamber 1314-5 swells closing off the fifth reservoir 1306E. This causes a fifth capillary burst valve 1328E to rupture, allowing sweat to flow into a sixth reservoir 1306F.

Analogous to that above, upon filling the sixth reservoir 1306F, sweat flows along a return channel 1312F associated with the sixth reservoir 1306F to a sixth hydrogel valve chamber 1314A-6. A hydrogel valve within the sixth hydrogel valve chamber 1314-6 swells closing off the sixth reservoir 1306F. This causes a sixth capillary burst valve 1328F to rupture, allowing sweat to flow into an outlet 1318.

FIG. 13B schematically illustrates that as the sweat works through the collection system comprised of reservoirs (e.g., six reservoirs in this example), the reservoirs fill sequentially/serially. Moreover, a first reservoir completely fills before the next reservoir begins to fill. Yet further, there is no backflow or contamination between the reservoirs, as the hydrogel valves and capillary burst valves cooperate to isolate each reservoir as the system moves sequentially on to the next reservoir.

Yet further, FIG. 13B illustrates that not every hydrogel valve or capillary burst valve needs to be identical. Rather, the internal pressures, reservoir size, channel dimensions, and other factors can alter the specific design parameters of each hydrogel valve and/or capillary burst valve. This is schematically illustrated by different sized hydrogel valve chambers in FIG. 13B.

Referring to FIG. 13C, an example shows a reservoir 1306 filling, and a hydrogel valve within a hydrogel valve chamber 1314A beginning to be wetted by sweat. Here, the hydrogel valve is at an early stage of swelling.

Referring to FIG. 13D, the hydrogel valve within the hydrogel valve chamber 1314A has swollen to the point of pushing the corresponding PDMS wall against an inlet of the corresponding reservoir 1306.

Referring to FIG. 13E, the hydrogel valve within the hydrogel valve chamber 1314A has swollen to the point of pushing the corresponding PDMS wall against an inlet of the corresponding reservoir 1306 sufficient to close off the reservoir 1306. FIG. 13E illustrates the “hammerhead” shape of the hydrogel valve in the closed state where the reservoir 1306 is effectively sealed closed from the remainder of the downstream reservoirs.

### Example Bio Sensor Device

With regard to FIG. 1-13B generally, a biosensor device is provided, according to aspects herein. The biosensor device includes a first layer that couples the biosensor device to skin of a user. Here, the first layer may comprise, for example the adhesive layer 112, FIG. 1, adhesive layer 212, FIG. 2, wicking layer 220, FIG. 2, etc. A microfluidic layer is coupled to the first layer. For instance, the microfluidic layer can be implemented as the microfluidics layer 116, FIG. 1, microfluidics layer 216, FIG. 2, microfluidic device 500, FIG. 5, etc. Moreover, the microfluidic layer may couple directly to the first layer, or couple via an intermediate/intervening layer, such as an electrode layer 114, FIG. 1, electrode layer 214, FIG. 2, etc. As noted more fully herein, e.g., with regard to FIG. 5, the microfluidic layer sequentially captures and stores liquid collected from the skin of the user into discrete reservoirs. The discrete reservoirs include a first reservoir that collects a first sample of the liquid before a second reservoir collects a second sample of the liquid. Moreover, the microfluidic layer includes at least one valve that prevents the second sample collected into the second reservoir from contaminating the first sample collected into the first reservoir.

A first sensor comprises laser induced graphene electrodes, wherein the first sensor facilitates monitoring of a biological state or condition associated with the liquid collected from the skin of the user. In this regard, electronics couple to the laser induced graphene electrodes of the first sensor that collects a real-time measurement of the biological state or condition.

In some configurations, the liquid collected from the skin of the user comprises sweat. Here, the first sensor implements a sweat sensor, and the electronics collects the real-time measurement by detecting a change in impedance across the laser induced graphene electrodes as sweat flows over the first sensor.

In some other configurations, the liquid collected from the skin of the user comprises sweat. Here, the first sensor comprises a sweat sensor. The laser induced graphene electrodes comprise a pair of parallel band electrodes that align along the first reservoir. The electronics collect real-time measurements by detecting a change in impedance across the laser induced graphene electrodes as sweat flows over the first sensor. Moreover, the electronics determine from the change in impedance measurements a flow rate of sweat into the first reservoir.

In yet some other configurations, the first sensor comprises a sweat sensor. Here, the first reservoir and the second reservoir each form a serpentine pattern, and the laser induced graphene electrodes of the first sensor comprise a first pair of parallel band electrodes that align along the serpentine pattern of the first reservoir. In this configuration, the biosensor device may further comprise a second sensor that implements a sweat sensor, where the second sensor comprises laser induced graphene electrodes implemented as a second pair of parallel band electrodes that align along the serpentine pattern of the second reservoir. Here, the second sensor facilitates monitoring of a biological state or condition associated with the liquid collected from the skin of the user. Optionally, the electronics collects a first real-time measurement by detecting a change in impedance across the first pair of parallel band electrodes as sweat flows over the first sensor and is collected into the first reservoir. Here, the electronics record a volumetric flow rate of sweat flowing into the first reservoir, and the electronics record a first chronological time stamp corresponding to when sweat flows into the first reservoir. Also, the electronics collect a second real-time measurement by detecting a change in impedance across the second pair of parallel band electrodes as sweat flows over the second sensor and is collected into the second reservoir. In this manner, the electronics record a volumetric flow rate of sweat flowing into the second reservoir and the electronics records a second chronological time stamp corresponding to when sweat flows into the second reservoir.

In still further configurations, the liquid collected from the skin of the user comprises sweat. Here, the first sensor comprises a sweat sensor, and the laser induced graphene electrodes comprise a pair of parallel band electrodes. The electronics collect real-time measurements by detecting a change in impedance across the laser induced graphene electrodes as sweat flows over the first sensor. Moreover, the pair of parallel band electrodes of the first sensor form a closed loop such that an initial resistance across the pair of electrodes is not infinite.

In still other embodiments, the laser induced graphene electrodes are formed on a polyimide film that that has been carbonized by a laser to form a graphite/graphene-like planar geometry.

In some instances, the first sensor comprises at least three laser induced graphene electrodes defining a working electrode, a reference electrode, and a counter electrode. Here, platinum is deposited on the counter electrode, Ag/AgCl is provided on the reference electrode, and the working electrode is platinized, functionalized with an amine binding chemistry, and is bonded to a biorecognition element.

In still some configurations, the at least one valve of the microfluidic layer that prevents the second sample collected into the second reservoir from contaminating the first sample collected into the first reservoir comprises a swelling hydrogel valve that contacts the sweat collected into the first reservoir, swells and collapses an inlet channel of the first reservoir. Optionally, the microfluidic layer comprises a Polydimethylsiloxane (PDMS) microfluidic layer, and the swelling hydrogel valve comprises a sodium acrylate hydrogel that is patterned in a channel of the microfluidic layer and is configured to swell when in contact with a liquid, closing off the channel. Moreover, optionally, the microfluidic layer comprises at least one passive flow control capillary burst valve (CBV) associated with each discrete reservoir that controls filling of the associated discrete reservoir. Still further, in some configurations, at least one capillary burst valve and at least one hydrogel valve cooperate to capture sweat into each reservoir.

### Example Bio Sensor Device

A biosensor device can also comprise a first layer that couples the biosensor device to skin of a user, and a microfluidic layer coupled to the first layer, analogous to that described above. In this configuration, the microfluidic layer sequentially captures and stores liquid collected from the skin of the user into discrete reservoirs. Additionally, the discrete reservoirs include a first reservoir that forms a serpentine pattern and collects a first sample of the liquid, and the discrete reservoirs include a second reservoir that forms a serpentine pattern and collects a first sample of the liquid. A first sensor comprises a first pair of laser induced graphene electrodes that align along the serpentine pattern of the first reservoir. Similarly, a second sensor comprises a second pair of laser induced graphene electrodes that align along the serpentine pattern of the second reservoir. Electronics are coupled to the first sensor and the second sensor. In this regard, the electronics collect real-time measurements by detecting a change in impedance across the first pair of laser induced graphene electrodes as sweat flows over the first sensor, and the electronics determine from the change in impedance measurements, a flow rate of sweat into the first reservoir. Also, the electronics collect real-time measurements by detecting a change in impedance across the second pair of laser induced graphene electrodes as sweat flows over the second sensor, and the electronics determine from the change in impedance measurements a flow rate of sweat into the second reservoir.

In some configurations, a third sensor is provided, which includes at least three laser induced graphene electrodes. For instance, a first electrode can define a working electrode, a second electrode can define a reference electrode, and a third electrode can define a counter electrode. Moreover, in some implementations, platinum is deposited on the counter electrode, Ag/AgCl is provided on the reference electrode, and the working electrode is platinized, functionalized with an amine binding chemistry, and is bonded to a biorecognition element.

Also, in some configurations, a swelling hydrogel valve that contacts the sweat collected into the first reservoir, swells and collapses an inlet channel of the first reservoir to prevent the second sample collected into the second reservoir from contaminating the first sample collected into the first reservoir. Optionally, the microfluidic layer can comprise a Polydimethylsiloxane (PDMS) microfluidic layer, and the swelling hydrogel valve comprises a sodium acrylate hydrogel that is patterned in a channel of the microfluidic layer and is configured to swell when in contact with a liquid, closing off the channel.

In yet some further configurations, the microfluidic layer comprises at least one passive flow control capillary burst valve (CBV) associated with each discrete reservoir that controls filling of the associated discrete reservoir.

Moreover, in some configurations, the biosensor device further comprises at least one passive flow control capillary burst valve (CBV) associated with each discrete reservoir that controls filling of the associated discrete reservoir, and a swelling hydrogel valve associated with each discrete reservoir that contacts the sweat collected into the associated discrete reservoir, swells and collapses an inlet channel thereof to prevent a sample collected into the associated discrete reservoir from contaminating samples collected into the other discrete reservoirs.

### Example Bio Sensor Device

In some configurations, a biosensor device comprises a first layer that couples the biosensor device to skin of a user, and a Polydimethylsiloxane (PDMS) microfluidic layer coupled to the first layer that collects sweat from the skin of the user, e.g., analogous to that described more fully herein. The microfluidic layer comprises an inlet and an array of reservoirs coupled to the inlet. Here, each reservoir of the array of reservoirs includes at least one passive flow control capillary burst valve (CBV) to control filling of the corresponding reservoir. Each reservoir of the array of reservoirs also includes a swelling hydrogel valve that contacts the sweat collected into the associated reservoir, swells and collapses an inlet channel thereof. Each reservoir fills in series such that a first one of the reservoirs fills before filling a second one of the reservoirs.

Also, the biosensor device can comprise a first sensor that monitors a biological state or condition associated with the liquid collected from the skin of the user, and electronics coupled to the first sensor that collects a real-time measurement of the biological state or condition.

In some configurations, each capillary burst valve is positioned after an inlet of the corresponding reservoir, and controls flow of sweat into the corresponding reservoir by introducing a sudden expansion in a channel of the corresponding reservoir, which confines a meniscus at an entrance of the expansion until a driving force can overcome an increased pressure barrier. Also, each reservoir comprises an air vent, wherein an additional capillary burst valve is positioned to prevent sweat exiting the corresponding reservoir through the associated air vent. Still further, each swelling hydrogel valve comprises a sodium acrylate hydrogel that is patterned in a microfluidic channel and is configured to swell when in contact with sweat, closing off the channel.

### Medical Adhesive

In order to secure the sweat patch device to the wearer, a medical-grade adhesive can be incorporated into the patch design. An appropriate medical-grade adhesive could be long lasting, e.g., to withstand 8 hours of vigorous activity, waterproof to resist lifting when in contact with sweat, and breathable to minimize irritation of the wearer's skin. Additionally, the adhesive needs to be flexible to conform to the curvature of the wearer's body and thin to not add additional bulkiness to the overall sweat patch device.

### Wicking Layer

In order to direct sweat into the microfluidic patch, a wicking layer may be provided. For instance, a shaped (e.g., star-shaped) wicking layer can be cut from nitrocellulose paper. Nitrocellulose paper may provide minimal potential for introducing contamination to the sweat samples and moderate flow-through rate. However, the time required to wet the wicking layer may result in a delay in readings. Moreover, the absorption rate can cause variations in the detected sweat rate. Additionally, any sweat wicking material will adsorb some level of sweat which will remain in the material, contaminating the time-stamp of later sweat.

To remove these unwanted side effects, the wicking layer can be implemented by using the capillary force between the user's skin and the sweat patch to wick sweat towards the inlet of the patch. That is, no physical wicking layer (e.g., no nitrocellulose paper wicking layer) is utilized.

Rather, a cutout can be provided in the adhesive layer. By way of example, a cutout in the form of a small circle can be provided in the adhesive layer, generally centered with the inlet(s) of the microfluidic layer(s). Using this design, adhesion to the wearer was increased while the variations in sweat rate data were reduced. Furthermore, the fill time/wear time of the sweat patch can be customized by increasing or decreasing the area of the cut out in the adhesion layer.

### Integration of LIG Sweat Rate Sensors into The Microfluidic

Initially the electrodes can have infinite resistance, but when sweat enters the channel, the sweat shorts the electrode leads. Further filling of the reservoir causes the series resistance of the electrodes to decrease. A calibration curve can be constructed, for instance, by plotting a measured series resistance the electrodes as a function of volume pumped (V*t).

In some implementations, the laser induced graphene electrode forms an open loop. In other embodiments, the laser induced graphene electrode forms a closed loop. An open loop can be useful where it is desirable to create a significant drop in impedance upon sweat entering the reservoir. On the other hand, the closed loop is still able to sensitively sense sweat as it shorts the electrodes. Additionally, as the LIG is closed, a fixed resistance can be measured before sweat has entered the reservoir. By measuring this resistance during fabrication, a quality assurance step can be introduced to ensure proper fabrication of the sweat sensor within the patch.

Before sweat enters the microfluidic, the impedance of the sweat rate sensors is extremely constant (e.g., ˜60 kΩ. When sweat enters the reservoir and comes in contact with the LIG electrode, it shorts the end of the serpentine electrode causing a small jump in impedance. As sweat continues to flow into the reservoir and over the LIG electrode, the impedance then decreases. The change in impedance is linear, e.g., where a constant volumetric flow rate is achieved. The volumetric flow rate is calculated by correlating the change in impedance to the volume of the sweat collection reservoir.

### Smart App

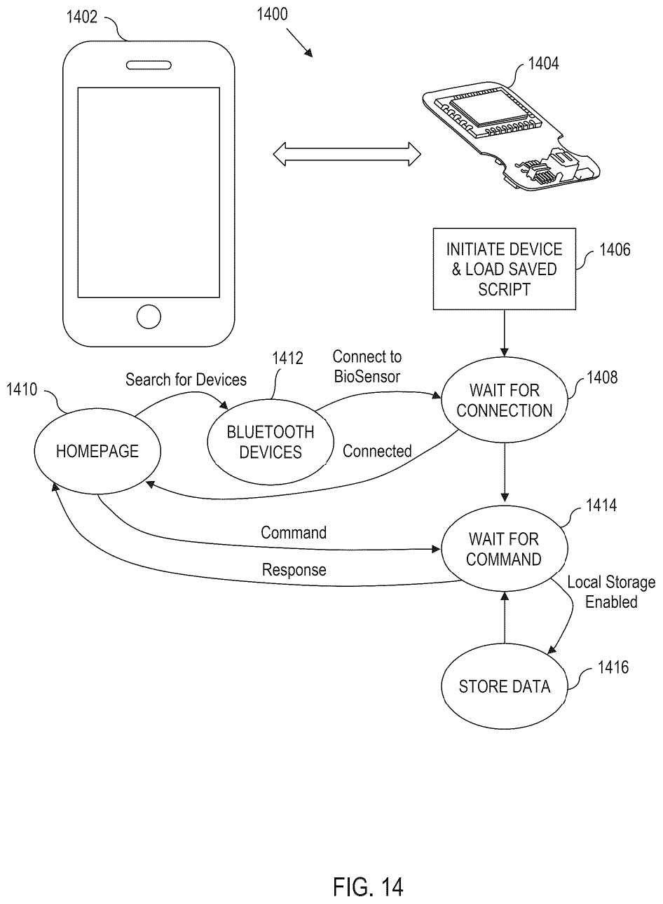

Referring to FIG. 14, a flow 1400 illustrates a smartphone 1402 running a custom smartphone app that communicates with electronics 1404, e.g., a potentiostat chip, which can be located within the reusable electronics (e.g., within the optional secondary device 104, FIG. 1). The custom smartphone app of the smartphone 1402 further comprises a graphical user interface to display data as described more fully herein.

In an example implementation, Bluetooth data is passed between the smartphone 1402 and electronics 1404, allowing the device to send commands to the potentiostat chip. In an example configuration, the software on the biosensor patch board works primarily as a passthrough command from a Bluetooth device to the potentiostat chip sensor.

The device electronics 1404 initializes and loads at 1406 the script currently stored in the potentiostat chip, then waits at 1408 for a command from the phone or PC. Here, a homepage at 1410 or other GUI feature of the smartphone 1402 can search for devices (e.g., Bluetooth devices) at 1412 to connect to the biosensor electronics 1404. The GUI of the smartphone 1402 can also send commands and receive responses, e.g., by waiting for commands at 1414 storing data at 1416, etc.

This approach allows the GUI of the smartphone 1402 to treat the biosensor board as a standalone potentiostat chip, giving the ability to load new scripts through the Bluetooth interface without any translation required between the devices and no firmware update for the biosensor board. In example embodiments, the smartphone 1402 has the ability to send commands, e.g., from a homepage at 1410 to store data onboard and retrieve the data, giving the ability to store data when a Bluetooth device is not available. The phone application has potentiostat chip commands available through push buttons and onboard storage commands through checkboxes. Data is then displayed on the phone in graph form as well as saved on the phone, e.g., in.csv format.

### Calculation of Collected Volume

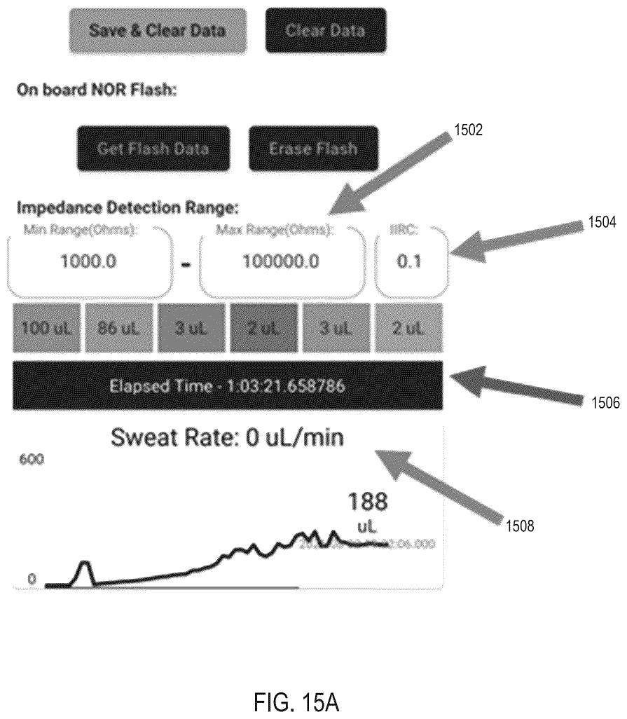

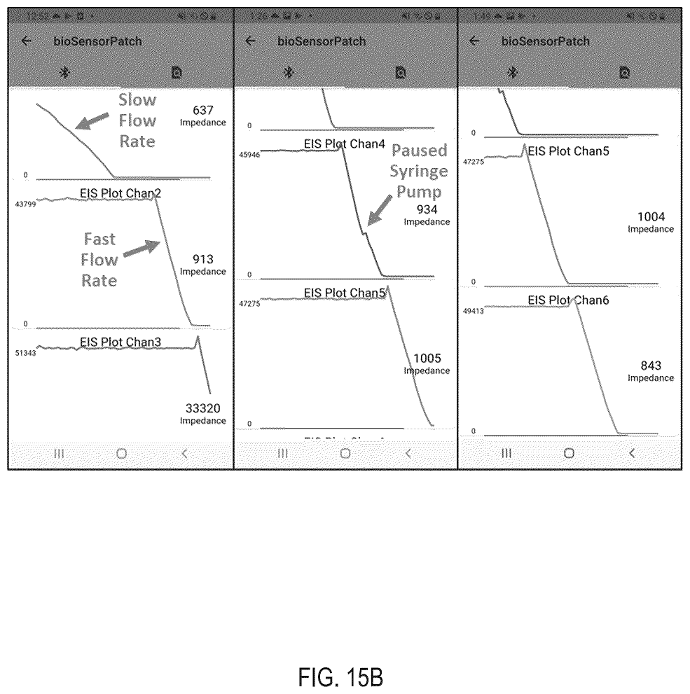

Referring to FIGS. 15A and 15B, aspects herein provide a method to calculate the collected volume of each of the different reservoirs. Upon starting, the app measures the impedance of the LIG electrodes which correlates to zero sweat collected. As the impedance decreases, it correlates to an increase in collected sweat. Editable text boxes 1502 allow the user to set the minimum and maximum range for the impedance measurement. While the impedance measurement will still be recorded if the values are outside the designated range, they will not be included in the sweat rate or accumulate sweat volume graph. This eliminates bad data, such as noise or if the channel shorts/breaks. Using an Infinite Impulse Response Coefficient (IIRC) 1504, the weighted average can be adjusted giving more importance on the newest values. Additionally, an elapse time 1506 can be added, which records how much time the sweat patch sensors have been running. In some embodiments, a sweat rate plot displays the accumulated collected sweat over time 1508. The sweat rate displays the total sweat accumulation over an interval such as a 5 minute interval Finally, the display of the graph shifts over time displaying the latest data. Note that each of the different screenshots of the app were taken at different times as the plots continuously update while running.

### Miscellaneous

According to aspects herein, a flexible and modular sweat collection patch is provided, which includes integrated biosensor capabilities. This wearable sweat patch incorporates a flexible electrode layer, microfluidic layer, and miniature electronics in a stacked structure. A long-wear medical adhesive can be used to attach the device to the wearer. The flexible electrode layer contains a sensitive sweat rate sensor as well as a biosensor platform. The microfluidic layer utilizes valves (e.g., passive valves) to direct the flow of sweat throughout the device and chronologically store sweat in discreet reservoirs, isolating the sweat samples for later extraction and analysis. The flexible electronic layer connects to a reusable portable electronic system, e.g., which may include a potentiostat system. The electronics wirelessly communicate to an app that displays the device data in real-time.

According to further aspects herein, a modular biosensor patch is provided. Utilizing capillary burst valves, the modular biosensor patch can effectively collect and chronologically store sweat into distinct reservoirs, which can then be extracted for post analysis. The six sweat collection reservoirs can store hundreds of nanoliters (e.g., up to ˜100 μL each reservoir in an illustrative example), giving the total capacity of the sweat patch close to ˜ 600 μL in this example.

A sensitive sweat rate sensor and modular biosensor platform is also provided, utilizing laser induced graphene which has a high surface area and tunable conductivity. The sweat rate sensor is operated by measuring the impedance of the LIG electrode, which decreases as sweat flows through the reservoir. This method for measuring sweat rate is extremely sensitive, provides real-time feedback into the user's sweat rate and provides a time-stamp for each of the sweat collection reservoirs.

Moreover, the biosensor capability can include an electrochemical biosensor, e.g., using lactate oxidase, as described more fully herein.

The compact electronic board employs a small potentiostat chip, allowing electrochemical scripts to be directly integrated into code. The phone app wirelessly communicates with the electronics and provides real-time feed-back of the user's sweat rate, collected sweat volume, and an amperometric response to lactate in the sweat.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

The corresponding structures, materials, acts, and equivalents of all means or step plus function elements in the claims below are intended to include any structure, material, or act for performing the function in combination with other claimed elements as specifically claimed. The description of the present invention has been presented for purposes of illustration and description, but is not intended to be exhaustive or limited to the invention in the form disclosed. Many modifications and variations will be apparent to those of ordinary skill in the art without departing from the scope and spirit of the invention. Aspects of the invention were chosen and described in order to best explain the principles of the invention and the practical application, and to enable others of ordinary skill in the art to understand the invention for various embodiments with various modifications as are suited to the particular use contemplated.

What is claimed is:

1. A biosensor device comprising:
a first layer that couples the biosensor device to skin of a user;
a microfluidic layer coupled to the first layer, wherein:
the microfluidic layer sequentially captures and stores liquid collected from the skin of the user into discrete reservoirs;
the discrete reservoirs include a first reservoir that collects a first sample of the liquid before a second reservoir collects a second sample of the liquid; and
the microfluidic layer includes at least one valve that prevents the second sample collected into the second reservoir from contaminating the first sample collected into the first reservoir;
a first sensor comprising laser induced graphene (LIG) electrodes, wherein the first sensor facilitates monitoring of a biological state or condition associated with the liquid collected from the skin of the user; and
electronics coupled to the laser induced graphene electrodes of the first sensor that collects a real-time measurement of the biological state or condition.

2. The biosensor device of claim 1, wherein:
the liquid collected from the skin of the user comprises sweat;
first sensor implements a sweat sensor;
the electronics collects the real-time measurement by detecting a change in impedance across the laser induced graphene electrodes as sweat flows over the first sensor.

3. The biosensor device of claim 1, wherein:
the liquid collected from the skin of the user comprises sweat;
the first sensor comprises a sweat sensor;
the laser induced graphene electrodes comprise a pair of parallel band electrodes that align along the first reservoir;
the electronics collect real-time measurements by detecting a change in impedance across the laser induced graphene electrodes as sweat flows over the first sensor; and
the electronics determine from the change in impedance measurements a flow rate of sweat into the first reservoir.

4. The biosensor device of claim 1, wherein:
the first sensor comprises a sweat sensor;
the first reservoir and the second reservoir each form a serpentine pattern; and
the laser induced graphene electrodes of the first sensor comprise a first pair of parallel band electrodes that align along the serpentine pattern of the first reservoir;
further comprising:
a second sensor that implements a sweat sensor, the second sensor comprising laser induced graphene electrodes implemented as a second pair of parallel band electrodes that align along the serpentine pattern of the second reservoir, wherein the second sensor facilitates monitoring of a biological state or condition associated with the liquid collected from the skin of the user.

5. The biosensor device of claim 4, wherein:
the electronics collect a first real-time measurement by detecting a change in impedance across the first pair of parallel band electrodes as sweat flows over the first sensor and is collected into the first reservoir;
the electronics record a volumetric flow rate of sweat flowing into the first reservoir;
the electronics record a first chronological time stamp corresponding to when sweat flows into the first reservoir;
the electronics collect a second real-time measurement by detecting a change in impedance across the second pair of parallel band electrodes as sweat flows over the second sensor and is collected into the second reservoir;
the electronics record a volumetric flow rate of sweat flowing into the second reservoir; and
the electronics record a second chronological time stamp corresponding to when sweat flows into the second reservoir.

6. The biosensor device of claim 1, wherein:
the liquid collected from the skin of the user comprises sweat;
the first sensor comprises a sweat sensor;
the laser induced graphene electrodes comprise a pair of parallel band electrodes;
the electronics collect real-time measurements by detecting a change in impedance across the laser induced graphene electrodes as sweat flows over the first sensor; and
the pair of parallel band electrodes of the first sensor form a closed loop such that an initial resistance across the pair of electrodes is not infinite.

7. The biosensor device of claim 1, wherein:
the laser induced graphene electrodes are formed on a polyimide film that that has been carbonized by a laser to form a graphite/graphene-like planar geometry.

8. The biosensor device of claim 1, wherein:
the first sensor comprises at least three laser induced graphene electrodes defining a working electrode, a reference electrode, and a counter electrode, wherein:
platinum is deposited on the counter electrode;
Ag/AgCl is provided on the reference electrode; and
the working electrode is platinized, functionalized with an amine binding chemistry, and is bonded to a biorecognition element.

9. The biosensor device of claim 1, wherein:
at least one valve of the microfluidic layer that prevents the second sample collected into the second reservoir from contaminating the first sample collected into the first reservoir comprises a swelling hydrogel valve that contacts the sweat collected into the first reservoir, swells and collapses an inlet channel of the first reservoir.

10. The biosensor device of claim 9, wherein:
the microfluidic layer comprises a Polydimethylsiloxane (PDMS) microfluidic layer; and
the swelling hydrogel valve comprises a sodium acrylate hydrogel that is patterned in a channel of the microfluidic layer and is configured to swell when in contact with a liquid, closing off the channel.

11. The biosensor device of claim 9, wherein:
the microfluidic layer comprises at least one passive flow control capillary burst valve (CBV) associated with each discrete reservoir that controls filling of the associated discrete reservoir.

12. The biosensor device of claim 11, wherein at least one capillary burst valve and at least one hydrogel valve cooperate to capture sweat into each reservoir.

13. A biosensor device comprising:
a first layer that couples the biosensor device to skin of a user;
a microfluidic layer coupled to the first layer, wherein the microfluidic layer sequentially captures and stores liquid collected from the skin of the user into discrete reservoirs, wherein:
the discrete reservoirs include a first reservoir that forms a serpentine pattern and collects a first sample of the liquid; and
the discrete reservoirs include a second reservoir that forms a serpentine pattern and collects a second sample of the liquid;
a first sensor comprising a first pair of laser induced graphene electrodes that align along the serpentine pattern of the first reservoir;
a second sensor comprising a second pair of laser induced graphene electrodes that align along the serpentine pattern of the second reservoir; and
electronics coupled to the first sensor and the second sensor, wherein:
the electronics collect real-time measurements by detecting a change in impedance across the first pair of laser induced graphene electrodes as sweat flows over the first sensor, and the electronics determine from the change in impedance measurements, a flow rate of sweat into the first reservoir; and
the electronics collect real-time measurements by detecting a change in impedance across the second pair of laser induced graphene electrodes as sweat flows over the second sensor, and the electronics determine from the change in impedance measurements, a flow rate of sweat into the second reservoir.

14. The biosensor device of claim 13 further comprising:
a third sensor having at least three laser induced graphene electrodes defining a working electrode, a reference electrode, and a counter electrode, wherein:
platinum is deposited on the counter electrode;
Ag/AgCl is provided on the reference electrode; and
the working electrode is platinized, functionalized with an amine binding chemistry, and is bonded to a biorecognition element.

15. The biosensor device of claim 13 further comprising:
a swelling hydrogel valve that contacts the sweat collected into the first reservoir, swells and collapses an inlet channel of the first reservoir to prevent the second sample collected into the second reservoir from contaminating the first sample collected into the first reservoir.

16. The biosensor device of claim 15, wherein:
the microfluidic layer comprises a Polydimethylsiloxane (PDMS) microfluidic layer; and
the swelling hydrogel valve comprises a sodium acrylate hydrogel that is patterned in a channel of the microfluidic layer and is configured to swell when in contact with a liquid, closing off the channel.

17. The biosensor device of claim 13, wherein:
the microfluidic layer comprises at least one passive flow control capillary burst valve (CBV) associated with each discrete reservoir that controls filling of the associated discrete reservoir.

18. The biosensor device of claim 13 further comprising:
at least one passive flow control capillary burst valve (CBV) associated with each discrete reservoir that controls filling of the associated discrete reservoir; and
a swelling hydrogel valve associated with each discrete reservoir that contacts the sweat collected into the associated discrete reservoir, swells and collapses an inlet channel thereof to prevent a sample collected into the associated discrete reservoir from contaminating samples collected into the other discrete reservoirs.

19. A biosensor device comprising:
a first layer that couples the biosensor device to skin of a user; and
a Polydimethylsiloxane (PDMS) microfluidic layer coupled to the first layer that collects sweat from the skin of the user, the microfluidic layer comprising:
an inlet; and
an array of reservoirs coupled to the inlet;
wherein:
each reservoir of the array of reservoirs includes at least one passive flow control capillary burst valve (CBV) to control filling of the corresponding reservoir;
each reservoir of the array of reservoirs includes a swelling hydrogel valve that contacts the sweat collected into the associated reservoir, swells and collapses an inlet channel thereof; and
each reservoir fills in series such that a first one of the reservoirs fills before filling a second one of the reservoirs.

20. The biosensor of claim 19 further comprising a first sensor that monitors a biological state or condition associated with the liquid collected from the skin of the user; and
electronics coupled to the first sensor that collects a real-time measurement of the biological state or condition.

21. The biosensor device of claim 19, wherein:
each capillary burst valve is positioned after an inlet of the corresponding reservoir, and controls flow of sweat into the corresponding reservoir by introducing a sudden expansion in a channel of the corresponding reservoir, which confines a meniscus at an entrance of the expansion until a driving force can overcome an increased pressure barrier;
each reservoir comprises an air vent, wherein an additional capillary burst valve is positioned to prevent sweat exiting the corresponding reservoir through the associated air vent; and
each swelling hydrogel valve comprises a sodium acrylate hydrogel that is patterned in a microfluidic channel and is configured to swell when in contact with sweat, closing off the channel.
